(19) United States
(12) Patent Application Publication
(10) Pub. No.: US 2024/0417472 A1
(43) Pub. Date: Dec. 19, 2024

(54) PD-1/TIM-3 BINDING PROTEINS FOR TREATMENT OF NSCLC and cHL

(71) Applicant: MEDIMMUNE, LLC, GAITHERSBURG, MD (US)

(72) Inventors: Natalia CEAICOVSCAIA, Gaithersburg, MD (US); Song CHO, Gaithersburg, MD (US); Emma DEAN, Cambridge (GB); Connor HALL, Gaithersburg, MD (US); Michele PETRUZZELLI, Cambridge (GB)

(21) Appl. No.: 18/671,167

(22) Filed: May 22, 2024

Related U.S. Application Data
(60) Provisional application No. 63/607,939, filed on Dec. 8, 2023.
(60) Provisional application No. 63/591,536, filed on Oct. 19, 2023.
(60) Provisional application No. 63/503,793, filed on May 23, 2023.

(51) Int. Cl. C07K 16/28; A61K 9/00; A61K 39/00; A61P 35/00

(52) U.S. Cl. CPC C07K 16/2818; A61K 9/0019; A61P 35/00; A61K 2039/505; A61K 2039/545; C07K 2317/24; C07K 2317/31; C07K 2317/41; C07K 2317/52; C07K 2317/565

(57) ABSTRACT

The disclosure relates to methods of treating non-small cell lung cancer (NSCLC) or classical Hodgkin's Lymphoma (cHL) by administering a bispecific antibody, that binds to Programmed Death-1 (PD-1) and T-cell immunoglobulin and mucin domain containing protein-3 (TIM-3), to a subject with NSCLC or cHL in an amount from about 70 mg to about 1500 mg.

### REFERENCE TO SEQUENCE LISTING

This application contains a sequence listing which is submitted electronically and is hereby incorporated by reference in its entirety. The sequence listing submitted herewith is contained in the XML filed created May 2, 2024 entitled “PDTM-210-WO-PCT_Sequence-Listing.xml” and is 43,117 bytes in size.

### FIELD

The present disclosure provides methods for treating NSCLC or cHL in a subject, comprising administering to the subject a bispecific binding protein that specifically binds to Programmed Death-1 (PD-1) and T-cell immunoglobulin and mucin domain containing protein-3 (TIM-3) in an amount from about 70 mg to about 1500 mg.

### BACKGROUND

Programmed Death-1 (PD-1) is an approximately 31 kD type I membrane protein that is a member of the extended CD28/CTLA4 family of T cell regulators (see Ishida et al., Induced Expression of PD-1, A Novel Member of the Immunoglobulin Gene Superfamily, Upon Programmed Cell Death, EMBO J. 1992, 11:3887-95). PD-1 is expressed on activated T cells, B cells, and monocytes and at low levels in natural killer (NK) T cells. PD-1 is a well-validated target for immune mediated therapy in oncology. Antagonistic inhibition of the PD-1/PD-L1 interaction increases T cell activation, enhancing recognition and elimination of tumor cells by the host immune system.

TIM-3 is a transmembrane protein of T lymphocytes (CD4+ and CD8+ T cells), other lymphocytes, myeloid cells, or other cell types in different tumors. TIM-3 is a mediator of phagocytosis of apoptotic cells and contributes to antigen cross presentation. TIM-3 is highly expressed on intra-tumoral T-cells and is associated with high levels of PD-1 (Thommen, D et al. Nature Med 2018, 24:994-1004). Dual expression of PD-1 & TIM-3 inversely correlates with T-cell function (Sakuishi, K et al. J. Exp. Med. 2010, 207 (10): 2187-2194).

### SUMMARY

In aspects, the present disclosure provides a method for treating non-small cell lung cancer (NSCLC) or classical Hodgkin Lymphoma (cHL) in a subject, comprising administering to the subject a bispecific binding protein that specifically binds to Programed Death-1 (PD-1) and T cell immunoglobulin and mucin domain-containing protein 3 (TIM-3) in an amount from about 70 mg to about 1500 mg, the bispecific binding protein comprising: a) a first binding domain that specifically binds to PD-1, wherein the first binding domain comprises a heavy chain variable domain comprising a HCDR1 having the amino acid sequence of SEQ ID NO: 4, a HCDR2 having the amino acid sequence of SEQ ID NO: 5, and a HCDR3 having the amino acid sequence of SEQ ID NO: 6, and a light chain variable domain comprising a LCDR1 having the amino acid sequence of SEQ ID NO: 10, a LCDR2 having the amino acid sequence of SEQ ID NO: 11 and a LCDR3 having the amino acid sequence of SEQ ID NO: 12; and b) a second binding domain that specifically binds to TIM-3, wherein the second binding domain comprises a heavy chain variable domain comprising a HCDR1 having the amino acid sequence of SEQ ID NO: 1, a HCDR2 having the amino acid sequence of SEQ ID NO: 2, and a HCDR3 having the amino acid sequence of SEQ ID NO: 3, and a light chain variable domain comprising a LCDR1 having the amino acid sequence of SEQ ID NO: 7, a LCDR2 having the amino acid sequence of SEQ ID NO: 8, and a LCDR3 having the amino acid sequence of SEQ ID NO: 9.

In aspects of the method, the amount of the bispecific binding protein administered is about 70 mg, about 150 mg, about 210 mg, about 450 mg, about 750 mg, about 800 mg, about 850 mg, about 900 mg, about 950 mg, about 1000 mg, about 1250 mg, or about 1500 mg. In aspects, the amount of the bispecific binding protein administered is about 750 mg. In aspects, the amount of the bispecific binding protein administered is about 1500 mg.

In aspects of the method, the bispecific binding protein is administered once per treatment cycle. In aspects, the treatment cycle is about 7 days, about 14 days, about 21 days, about 28 days, or about 35 days. In aspects, the treatment cycle is about 21 days. In aspects, the treatment cycle is repeated for up to 35 cycles.

In aspects of the method, the second binding domain that specifically binds to TIM-3 specifically binds to the C′C″ and DE loops of the immunoglobulin variable (IgV) domain of TIM-3. In aspects, the second binding domain that specifically binds to TIM-3 specifically binds to epitopes on the IgV domain of TIM-3 and the epitopes comprises N12, L47, R52, D53, V54, N55, Y56, W57, W62, L63, N64, G65, D66, F67, R68, K69, D71, T75, and E77 of TIM-3 (SEQ ID NO: 29).

In aspects of the method, the bispecific binding protein is administered to the subject as a monotherapy. In aspects, the bispecific binding protein is administered by an intravenous infusion (IV).

In aspects of the method, the subject has not received a prior line of systemic therapy. In aspects, the subject has not received a prior line of immuno-oncology (IO) therapy. In aspects, the subject has previously received a chemotherapy. In aspects, the subject has previously received an IO therapy. In aspects, the IO therapy is an anti-PD-1/PD-LI therapy. In aspects, the IO therapy is an IO therapy other than an anti-PD-1/PD-L1 therapy.

In aspects of the method, the subject has IO acquired resistance. In aspects, the subject has acquired resistance to an anti-PD1 and/or anti-PD-L1 immuno-oncology therapy. In aspects, the subject has a radiologically documented tumor progression or clinical deterioration following initial treatment with an anti-PD-1/PD-L1 therapy for a minimum of 3-6 months, as monotherapy or in combination with chemotherapy, and had signs of initial clinical benefit, i.e. disease stabilization or regression.

In aspects of the method, the NSCLC or cHL comprises a NSCLC or cHL cell which expresses PD-L1.

In aspects of the method, the first binding domain of the bispecific binding protein that specifically binds to PD-1 comprises a heavy chain variable domain having the amino acid sequence of SEQ ID NO: 19 and a light chain variable domain having the amino acid sequence of SEQ ID NO:21. In aspects, the first binding domain of the bispecific binding protein that specifically binds to PD-1 comprises a heavy chain variable domain having an amino acid sequence that is at least 90% identical to the amino acid sequence of SEQ ID NO: 19 and a light chain variable domain having an amino acid sequence that is at least 90% identical to the amino acid sequence of SEQ ID NO:21.

In aspects of the method, the second binding domain of the bispecific binding protein that specifically binds to TIM-3 comprises a heavy chain variable domain having the amino acid sequence of SEQ ID NO: 14 and a light chain variable domain having the amino acid sequence of SEQ ID NO:17. In aspects, the second binding domain of the bispecific binding protein that specifically binds to TIM-3 comprises a heavy chain variable domain having an amino acid sequence that is at least 90% identical to the amino acid sequence of SEQ ID NO: 14 and a light chain variable domain having an amino acid sequence that is at least 90% identical to the amino acid sequence of SEQ ID NO:17.

In aspects of the method, the bispecific binding protein comprises a first heavy chain comprising the amino acid sequence of SEQ ID NO: 20, a first light chain comprising the amino acid sequence of SEQ ID NO: 22, a second heavy chain comprising the amino acid sequence of SEQ ID NO: 15, and a first light chain comprising the amino acid sequence of SEQ ID NO: 18.

In aspects of the method, the bispecific binding protein is a human or humanized bispecific antibody or antigen-binding fragment thereof. In aspects, the bispecific binding protein comprises a variant Fc region. In aspects, the variant Fc region of the bispecific binding protein comprises at least one substitution selected from 221K, 221Y, 225E, 225K, 225W, 228P, 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235E, 235F, 236E, 237L, 237M, 237P, 239D, 239E, 239N, 239Q, 239F, 239T, 239H, 239Y, 2401, 240A, 240T, 240M, 241W, 241L, 241Y, 241E, 241R, 243W, 243L 243Y, 243R, 243Q, 244H, 245A, 247L, 247V, 247G, 250E, 250Q, 251F, 252L, 252Y, 254S, 254T, 255L, 256E, 256F, 256M, 257C, 257M, 257N, 262I, 262A, 262T, 262E, 2631, 263A, 263T, 263M, 264L, 2641, 264W, 264T, 264R, 264F, 264M, 264Y, 264E, 265A, 265G, 265N, 265Q, 265Y, 265F, 265V, 2651, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 2961, 296H, 296G, 297S, 297D, 297E, 298A, 298H, 2981, 298T, 298F, 2991, 299L, 299A, 299S, 299V, 299H, 299F, 299E, 3051, 308F, 313F, 316D, 318A, 318S, 320A, 320S, 322A, 322S, 325Q, 325L, 3251, 325D, 325E, 325A, 325T, 325V, 325H, 326A, 326D, 326E, 326G, 326M, 326V, 327G, 327W, 327N, 327L, 328S, 328M, 328D, 328E, 328N, 328Q, 328F, 3281, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 333A, 333D, 333G, 333Q, 333S, 333V, 334A, 334E, 334H, 334L, 334M, 334Q, 334V, 334Y, 339T, 370E, 370N, 378D, 392T, 396L, 416G, 419H, 421K, 428L, 428F, 433K, 433L, 434A, 434W, 434Y, 436H, 440Y and 443W as numbered by the EU index as set forth in Kabat. In aspects, the variant Fc region of the bispecific binding protein comprises one or more amino acid substitutions at positions selected from 428 and 434 as numbered by the EU index as set forth in Kabat. In aspects, the variant Fc region of the bispecific binding protein comprises one or more amino acid substitutions selected from 428L, 428F, 434A, 424F, 434W, and 434Y. In aspects, the variant Fc region of the bispecific binding protein comprises a YTE mutation. In aspects, the Fc variant region of the bispecific binding protein comprises a L234F/L235E/P331S triple mutation (TM).

In aspects of the method, the Fc region of the bispecific binding protein is aglycosylated. In aspects, the Fc region of the bispecific binding protein is deglycosylated. In aspects, the Fc region of the bispecific binding protein has reduced fucosylation or is afucosylated.

In aspects of the method, the bispecific binding protein comprises a kappa light chain constant region. In aspects, the bispecific binding protein comprises a lambda light chain constant region.

In aspects of the method, the bispecific binding protein is an antibody. In aspects, the antibody is an IgG antibody. In aspects, the antibody is an IgG1 antibody. In aspects, the antibody is humanized.

In aspects, the cancer is non-small cell lung cancer (NSCLC). In aspects, the NSCLC is advanced or metastatic. In aspects, the NSCLC is squamous or non-squamous NSCLC. In aspects, the cancer is cHL. In aspects, the subject has a PD-L1 tumor proportion score of greater than or equal to 1%. In aspects, the subject has a PD-L1 tumor proportion score of greater than or equal to 50%. In aspects, the cHL patient is IO exposed.

In aspects of the method, the subject is checkpoint inhibitor (CPI) naïve.

In aspects, the present disclosure also provides a pharmaceutical composition comprising a bispecific binding protein that specifically binds to PD-1 and TIM-3 in an amount from about 70 mg to about 1500 mg, the bispecific binding protein comprising: a) a first binding domain that specifically binds to PD-1, wherein the first binding domain comprises a heavy chain variable domain comprising a HCDR1 having the amino acid sequence of SEQ ID NO: 4, a HCDR2 having the amino acid sequence of SEQ ID NO: 5, and a HCDR3 having the amino acid sequence of SEQ ID NO: 6, and a light chain variable domain comprising a LCDR1 having the amino acid sequence of SEQ ID NO: 10, a LCDR2 having the amino acid sequence of SEQ ID NO: 11 and a LCDR3 having the amino acid sequence of SEQ ID NO: 12; and b) a second binding domain that specifically binds to TIM-3, wherein the second binding domain comprises a heavy chain variable domain comprising a HCDR1 having the amino acid sequence of SEQ ID NO: 1, a HCDR2 having the amino acid sequence of SEQ ID NO: 2, and a HCDR3 having the amino acid sequence of SEQ ID NO: 3, and a light chain variable domain comprising a LCDR1 having the amino acid sequence of SEQ ID NO: 7, a LCDR2 having the amino acid sequence of SEQ ID NO: 8, and a LCDR3 having the amino acid sequence of SEQ ID NO: 9.

In aspects of the pharmaceutical composition, the pharmaceutical composition comprises about 70 mg, about 150 mg, about 210 mg, about 450 mg, about 750 mg, about 800 mg, about 850 mg, about 900 mg, about 950 mg, about 1000 mg, about 1250 mg, or about 1500 mg bispecific binding protein. In aspects, the pharmaceutical composition comprises about 750 mg bispecific binding protein. In aspects, the pharmaceutical composition comprises about 1500 mg bispecific binding protein.

In aspects of the pharmaceutical composition, the first binding domain of the bispecific binding protein that specifically binds to PD-1 comprises a heavy chain variable domain having the amino acid sequence of SEQ ID NO:19 and a light chain variable domain having the amino acid sequence of SEQ ID NO:21. In aspects, the first binding domain of the bispecific binding protein that specifically binds to PD-1 comprises a heavy chain variable domain having an amino acid sequence that is at least 90% identical to the amino acid sequence of SEQ ID NO:19 and a light chain variable domain having an amino acid sequence that is at least 90% identical to the amino acid sequence of SEQ ID NO:21.

In aspects of the pharmaceutical composition, the second binding domain of the bispecific binding protein that specifically binds to TIM-3 comprises a heavy chain variable domain having the amino acid sequence of SEQ ID NO: 14 and a light chain variable domain having the amino acid sequence of SEQ ID NO: 17. In aspects, the second binding domain of the bispecific binding protein that specifically binds to TIM-3 comprises a heavy chain variable domain having an amino acid sequence that is at least 90% identical to the amino acid sequence of SEQ ID NO:14 and a light chain variable domain having an amino acid sequence that is at least 90% identical to the amino acid sequence of SEQ ID NO:17.

In aspects of the pharmaceutical composition, the bispecific binding protein comprises a first heavy chain comprising the amino acid sequence of SEQ ID NO: 20, a first light chain comprising the amino acid sequence of SEQ ID NO: 22, a second heavy chain comprising the amino acid sequence of SEQ ID NO: 15, and a first light chain comprising the amino acid sequence of SEQ ID NO: 18.

In aspects, the present disclosure also provides a kit comprising a pharmaceutical composition as described herein. In aspects, the kit further comprises instructions for administering the pharmaceutical composition.

In aspects, the present disclosure provides a pharmaceutical composition as described herein for use in treating NSCLC or cHL. In aspects, the cancer is non-small cell lung cancer (NSCLC). In aspects, the NSCLC is advanced or metastatic. In aspects, the NSCLC is squamous or non-squamous NSCLC. In aspects the NSCLC or cHL has a PD-L1 tumor proportion score of greater than or equal to 1%. In aspects, the NSCLC or cHL has a PD-L1 tumor proportion score of greater than or equal to 50%. In aspects, the NSCLC or cHL patient has not previously been treated with a checkpoint inhibitor.

In aspects, the bispecific binding protein is AZD7789.

### BRIEF DESCRIPTION OF THE DRAWINGS

The following drawings form part of the present specification and are included to further demonstrate exemplary aspects of the present disclosure.

FIG. 1 is a waterfall plot of post-baseline scans of 19 subjects as described in Example 7. Plots are for the best percentage change from baseline of the target lesion size. For the plot: PD—progressive disease, NE—not evaluable, SD—stable disease, PR—partial response.

FIG. 2 is a plot of PD-1 receptor occupancy over time for AZD7789 doses of 225 mg, 750 mg, 1500 mg and 2000 mg as described in Example 9. As shown, durable receptor occupancy of greater than 90% is achieved with each dose.

FIG. 3A is a waterfall plot of post-baseline scans of 44 subjects as described in Example 9. Plots are for the best percentage change from baseline of the target lesion size. FIG. 3B is a plot of the duration of exposure to AZD7789 for each subject measured in weeks.

FIG. 4 is a box plot of T cell clonal expansion for treatments with AZD7789 and total expanded T cell clones and newly expanded T cell clones as described in Example 9. Dosing for each treatment is shown in the figure. The following abbreviations are used: BID, twice per day; C2D1, Cycle 2 Day 1; CD8, cluster of differentiation 8; IV, intravenously; PD-1, programmed cell death-1; Q3W, every 3 weeks; Q4W, every 4 weeks; RO, receptor occupancy; TCR, T cell receptor.

FIG. 5A is a plot of the duration of exposure to AZD7789 measured in weeks for the 32 subjects analyzed in Example 10. FIG. 5B is a waterfall plot of post-baseline scans of the 32 subjects. Plots are for the best percentage change from baseline of the target lesion size. The objective response of each subject is indicated as shown in the plot.

FIG. 6 shows full body scan images at both pre-treatment baseline and after 3 cycles of treatment with AZD7789 as described in Example 10. The abbreviations used are: ABVD, doxorubicin, bleomycin, vinblastine, dacarbazine; ASCT, autologous hematopoietic stem cell transplantation; Bv, brentuximab vedotin; Bv-AVD, Bv plus doxorubicin, vinblastine, and dacarbazine; C1D1, cycle 1 day 1; Cami-T, camidanlumab tesirine; CR, complete response; Gemox, gemcitabine and oxaliplatin; GVD, gemcitabine, vinorelbine, pegylated liposomal doxorubicin; ICE, ifosfamide, carboplatin, etoposide; n/a, not available; PD, progressive disease; PD-1, programmed cell death-1; PR, partial response; r/r cHL, relapsed or refractory classical Hodgkin lymphoma; and TIGIT, T cell immunoreceptor with immunoglobulin and ITIM domain.

### DETAILED DESCRIPTION

### Definitions

Unless otherwise defined herein, scientific, and technical terms used in the present disclosure shall have the meanings that are commonly understood by one of ordinary skill in the art. Further, unless otherwise required by context, singular terms shall include pluralities and plural terms shall include the singular.

As used herein, “a” or “an” may mean one or more. As used herein, when used in conjunction with the word “comprising,” the words “a” or “an” may mean one or more than one. As used herein, “another” or “a further” may mean at least a second or more.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited elements or method steps.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% or higher variability (either “greater than” or “less than” the indicated value), depending on the situation. In some aspects, one of skill in the art will understand the level of variability indicated by the term “about,” due to the context in which it is used herein. It should also be understood that use of the term “about” also includes the specifically recited value.

The use of the term “for example” and its corresponding abbreviation “e.g.,” (whether italicized or not) means that the specific terms recited are representative examples and aspects of the disclosure that are not intended to be limited to the specific examples referenced or cited unless explicitly stated otherwise.

Ranges provided herein, of any type, include all values within a particular range described and values about an endpoint for a particular range. As used herein, “between” is a range inclusive of the ends of the range. For example, a number between x and y explicitly includes the numbers x and y, and any numbers that fall within x and y.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus. Those of skill in the art, however, would appreciate that the locations of the domains in a naturally occurring antibody can be modified in certain antibody-like binding protein formats without a loss of antigen-binding capability. Classes of human light chains are termed kappa and lambda light chains.

Where not expressly stated, and unless the context indicates otherwise, the term “antibody” includes monospecific, bispecific, or multi-specific antibodies, as well as a single chain antibody. In some aspects, the antibody is a bispecific antibody. The term “bispecific antibodies” refers to antibodies that bind to two different epitopes. The epitopes can be on the same target antigen or can be on different target antigens.

In some aspects, the light chain constant region is a kappa chain. In some aspects, light chain constant region is a lambda chain.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “antibody fragment” refers to a portion of an intact or full-length chain or an antibody, generally the target binding or variable region. Examples of antibody fragments include, but are not limited to, F_{ab}, F_{ab′}, F_{(ab′)2} and Fᵥ fragments. As used herein, the term “functional fragment” is generally synonymous with “antibody fragment,” and with respect to antibodies, can refer to antibody fragments such as Fᵥ, F_{ab}, F_{(ab′)2}.

Reference to the numbering of amino acid residues described herein is performed according to the EU numbering system (also described in Kabat et al, Sequences of Proteins of Immunological Interest, 5th Ed. Public Health Service, National Institutes of Health, Bethesda, MD. (1991)).

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat EA & Wu TT (1971) Ann NY Acad Sci 190:382-391 and Kabat EA et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some aspects of the present disclosure, the CDRs of the antibodies described herein have been determined according to the Kabat numbering scheme.

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |
| | | (Kabat Numbering) | |
| H1 | H31-H35 | H26-H35 | H26-H32 |
| | | (Chothia Numbering) | |
| H2 | H50-H65 | H50-H58 | H52-H56 |
| H3 | H95-H102 | H95-H102 | H95-H102 |

The term “human antibody,” as used herein, includes antibodies having variable and constant regions substantially corresponding to human germline immunoglobulin sequences. In some aspects, human antibodies are produced in non-human mammals, including, but not limited to, rodents, such as mice and rats, and lagomorphs, such as rabbits. In other aspects, human antibodies are produced in hybridoma cells. In still other aspects, human antibodies are produced recombinantly. In some aspects, the bispecific binding protein is a human or humanized antibody.

The term “antigen” or “target antigen” as used herein refers to a molecule or a portion of a molecule that is capable of being recognized by and bound by binding proteins of the disclosure. The target antigen is capable of being used in an animal to produce antibodies capable of binding to an epitope of that antigen. A target antigen may have one or more epitopes.

The term “epitope” as used herein refers to a region or structural element of an antigen that is recognized and bound by a binding protein of the disclosure. More precisely, the epitope is the specific structure that is bound by the CDRs of the binding protein. Epitopes can comprise protein structural elements, carbohydrates or even portions of lipid structures found in membranes. A binding protein is said to specifically bind an antigen when it preferentially recognizes its antigen target in a complex mixture of proteins and/or macromolecules. The term “specifically binds” refers to a binding protein that specifically binds to a molecule or a fragment thereof (e.g., antigen). A binding protein that specifically binds a molecule or a fragment thereof may bind to other molecules with lower affinity as determined by, for example, immunoassays, BIAcore, or other assays known in the art. In particular, antibodies or fragments that specifically bind to at least one molecule or a fragment thereof can compete off molecules that bind non-specifically. The present disclosure specifically encompasses antibodies with multiple specificities (e.g., an antibody with specificity for two or more discrete antigens. For example, a bispecific antibody can bind to two adjacent epitopes on a single target antigen, or can bind to two different antigens.

The term “native Fc” as used herein refers to a molecule comprising the sequence of a non-antigen binding fragment resulting from digestion of an antibody or produced by other means, whether in monomeric or multimeric form, and can contain the hinge region. The original immunoglobulin source of the native Fc is preferably of human origin and can be any of the immunoglobulins. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a native Fc is a disulfide-bonded dimer resulting from papain digestion of an IgG. The term “native Fc” as used herein is generic to the monomeric, dimeric, and multimeric forms.

The term “Fc variant” as used herein refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. Furthermore, a native Fc comprises regions that can be removed or mutated to produce an Fc variant to alter certain residues that provide structural features or biological activity that are not required for the binding proteins of the disclosure. Thus, the term “Fc variant” comprises a molecule or sequence that lacks one or more native Fc sites or residues, or in which one or more Fc sites or residues has been modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The term “Fc domain” as used herein encompasses native Fc and Fc variants and sequences as defined above. As with Fc variants and native Fc molecules, the term “Fc domain” includes molecules in monomeric or multimeric form, whether digested from whole antibody or produced by other means.

The term “treating” or “treatment” refers to administering a compound or

pharmaceutical composition to a subject in order to effect an alteration or improvement of a disease, disorder, or condition in the subject. The terms “treatment” or “treat” as used herein may refer to both therapeutic treatment and prophylactic or preventative measures. Those in need of treatment include subjects having a disease or condition as well as those prone to having disease or condition or those for which a disease or condition is to be prevented.

The term “dose” means a specified quantity of a compound or pharmaceutical agent provided in a single administration, or in a specified time period. In some aspects, a dose can be administered in two or more boluses, tablets, or injections. For example, in some aspects, where subcutaneous administration is desired, the desired dose may require a volume not easily accommodated by a single injection. In such aspects, two or more injections can be used to achieve the desired dose. In some aspects, a dose can be administered in two or more injections to minimize injection site reaction in an individual. In other aspects, the compound or pharmaceutical agent is administered by infusion over an extended period of time or continuously. Doses can be stated as the amount of pharmaceutical agent per hour, day, week or month.

The terms “subject”, “individual” and “patient” are used interchangeably herein to refer to a mammalian subject. In one aspect the “subject” is a human, domestic animals, farm animals, sports animals, and zoo animals, e.g., humans, non-human primates, dogs, cats, guinea pigs, rabbits, rats, mice, horses, cattle, etc. In one aspect, the subject is a cynomolgus monkey (Macaca fascicularis). In a preferable aspect, the subject is a human. In methods of the disclosure, the subject may not have been previously diagnosed as having NSCLC or cHL. Alternatively, the subject may have been previously diagnosed as having NSCLC or cHL. The subject may also be one who exhibits disease risk factors, or one who is asymptomatic for NSCLC or cHL. The subject may also be one who is suffering from NSCLC or cHL In one aspect, the subject has been previously administered a cancer therapy. A subject who is “checkpoint inhibitor naïve” or “CPI naïve” means a subject whose cancer has not previously been treated with a checkpoint inhibitor (CPI).

The term “efficacy” means the ability to produce a desired effect. A “therapeutically effective dose” or “therapeutic dose” is an amount sufficient to effect desired clinical results (i.e., achieve therapeutic efficacy). A therapeutically effective dose can be administered in one or more administrations.

The term “side effects” means physiological disease and/or conditions attributable to a treatment other than the desired effects. In some aspects, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality. A “disease” or “condition” refers to any condition that would benefit from treatment using the methods of the disclosure. “Disease” and “condition” are used interchangeably herein and include chronic and acute disorders or diseases, including those pathological conditions that predispose a patient to the disorder in question. In some aspects, the disease is non-small cell lung cancer (NSCLC) or classical Hodgkin's Lymphoma (cHL). In some aspects, the NSCLC is advanced or metastatic. In some aspects, the advanced NSCLC is stage III or stage IV NSCLC.

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

The terms “pharmaceutical composition” or “therapeutic composition” as used herein refer to a compound or composition capable of inducing a desired therapeutic effect when properly administered to a subject. In some aspects, the disclosure provides a pharmaceutical composition comprising a pharmaceutically acceptable carrier and a therapeutically effective amount of binding proteins of the disclosure.

The terms “pharmaceutically acceptable carrier” or “physiologically acceptable carrier” as used herein refer to one or more formulation materials suitable for accomplishing or enhancing the delivery of one or more binding proteins of the disclosure.

### Methods of Treating NSCLC or cHL

The disclosure relates to a method for treating a non-small cell lung cancer (NSCLC) or classical Hodgkin lymphoma (cHL) in a subject comprising administering to the subject a bispecific binding protein (e.g., AZD7789) that specifically binds to PD-1 and TIM-3 (e.g., AZD7789) in an amount from about 70 mg to about 1500 mg. The disclosure also provides compositions, including pharmaceutical compositions, and kits, comprising such bispecific protein.

As used herein, bispecific binding proteins have binding specificities for at least two independent antigens (or targets) or different epitopes within the same antigen. Exemplary bispecific binding proteins may bind to two different epitopes of a target, or may bind two different targets. Other such binding proteins may combine a first target binding site with a second binding site for another target. In some aspects, the binding protein is a bispecific antibody.

In some aspects, bispecific antibodies provide additive and/or synergistic therapeutic effects derived from targeting two antigens simultaneously, with the administration of a single manufactured molecule.

AZD7789 was constructed on the backbone of the DuetMab molecule. The DuetMab design is described in Mazor et al., MAbs. 7(2): 377-389, (2015 March-April 2015), which is hereby incorporated by reference in its entirety. The “DuetMab,” design includes knobs-into-holes (KIH) technology for heterodimerization of 2 distinct heavy chains and increases the efficiency of cognate heavy and light chain pairing by replacing the native disulfide bond in one of the CH1-CL interfaces with an engineered disulfide bond. The Fc domain of AZD7789 carries the triple mutations (TM) (L234F, L235E and P331S) designed to reduce Fc-mediated immune effector functions (Oganesyan et al, Acta Crystallogr D Biol Crystallogr, 2008, 64 (Pt 6): 700-704). AZD7789 includes anti-PD-1 and anti-TIM-3 Fabs, engineered interchain disulfide in the anti-TIM-3 CH1-CL interface and knob-into-hole IgG1-TM Fc. AZD7789 includes a knob mutation in the heavy chain comprising a variable region that binds to TIM-3 and the hole mutation in the heavy chain comprising a variable region that binds to PD-1.

As used herein the term “AZD7789” refers to an anti-PD-1/TIM-3 bispecific antibody that comprises the heavy chain of SEQ ID NO: 15 and the light chain of SEQ ID NO: 18 (TIM-3) and the heavy chain of SEQ ID NO: 20 and the light chain of SEQ ID NO: 22 (PD-1). AZD7789 is described in U.S. Pat. No. 11,279,759, which is incorporated by reference herein in its entirety. As used herein the term “sabestomig” refers to AZD7789, and are used interchangeably.

In some aspects, the bispecific binding protein comprises: a) a first binding domain that specifically binds to PD-1, wherein the first binding domain comprises a heavy chain variable domain comprising a HCDR1 having the amino acid sequence of SEQ ID NO: 4, a HCDR2 having the amino acid sequence of SEQ ID NO: 5, and a HCDR3 having the amino acid sequence of SEQ ID NO: 6, and a light chain variable domain comprising a LCDR1 having the amino acid sequence of SEQ ID NO: 10, a LCDR2 having the amino acid sequence of SEQ ID NO: 11 and a LCDR3 having the amino acid sequence of SEQ ID NO: 12; and b) a second binding domain that specifically binds to TIM-3, wherein the second binding domain comprises a heavy chain variable domain comprising a HCDR1 having the amino acid sequence of SEQ ID NO: 1, a HCDR2 having the amino acid sequence of SEQ ID NO: 2, and a HCDR3 having the amino acid sequence of SEQ ID NO: 3, and a light chain variable domain comprising a LCDR1 having the amino acid sequence of SEQ ID NO: 7, a LCDR2 having the amino acid sequence of SEQ ID NO: 8, and a LCDR3 having the amino acid sequence of SEQ ID NO: 9. In some aspects, the bispecific binding protein is AZD7789.

In some aspects, the first binding domain that specifically binds to PD-1 comprises a heavy chain variable domain having the amino acid sequence of SEQ ID NO: 19 and a light chain variable domain having the amino acid sequence of SEQ ID NO:21.

In some aspects, the first binding domain that specifically binds to PD-1 comprises a heavy chain variable domain having an amino acid sequence that is at least 90, 91, 92, 93, 94, 95, 96, 97, 98, or 99% identical to the amino acid sequence of SEQ ID NO: 19. In some aspects, the first binding domain that specifically binds to PD-1 comprises a light chain variable domain having an amino acid sequence that is at least 90, 91, 92, 93, 94, 95, 96, 97, 98, or 99% identical to the amino acid sequence of SEQ ID NO: 21.

In some aspects, the second binding domain that specifically binds to TIM-3 comprises a heavy chain variable domain having the amino acid sequence of SEQ ID NO: 14 and a light chain variable domain having the amino acid sequence of SEQ ID NO:17.

In some aspects, the second binding domain that specifically binds to TIM-3 comprises a heavy chain variable domain having an amino acid sequence that is at least 90, 91, 92, 93, 94, 95, 96, 97, 98, or 99% identical to the amino acid sequence of SEQ ID NO: 14. In some aspects, the second binding domain that specifically binds to TIM-3 comprises a light chain variable domain having an amino acid sequence that is at least 90, 91, 92, 93, 94, 95, 96, 97, 98, or 99% identical to the amino acid sequence of SEQ ID NO: 17.

In some aspects, the first binding domain that specifically binds to PD-1 comprises a heavy chain is encoded by a nucleic acid sequence that is at least 90, 91, 92, 93, 94, 95, 96, 97, 98, or 99% identical to SEQ ID NO: 20 and a light chain is encoded by a nucleic acid sequence that is at least 90, 91, 92, 93, 94, 95, 96, 97, 98, or 99% identical to SEQ ID NO: 22.

In some aspects, the second binding domain that specifically binds to TIM-3 comprises a heavy chain that is encoded by a nucleic acid sequence that is at least 90, 91, 92, 93, 94, 95, 96, 97, 98, or 99% identical to SEQ ID NO: 15 and a light chain that is encoded by a nucleic acid sequence that is at least 90, 91, 92, 93, 94, 95, 96, 97, 98, or 99% identical to SEQ ID NO: 18.

In some aspects, the second binding domain that specifically binds to TIM-3 specifically binds to the C′C″ and DE loops of the IgV domain of TIM-3. In some aspects, the second binding domain specifically binds to epitopes on the IgV domain of TIM-3 and the epitopes comprises N12, L47, R52, D53, V54, N55, Y56, W57, W62, L63, N64, G65, D66, F67, R68, K69, D71, T75, and E77 of TIM-3 (SEQ ID NO: 29). The C′C″ loop of TIM-3 involves amino acids after beta strand C′ and before beta strand C″, for example, from amino acids 50 to 54. The DE loop consists of amino acids from 64 to 73, while the CC′ loop and FG loop comprise amino acids 35 to 43 and 92 to 99, respectively.

In some aspects, the Fc region is or includes a domain that is one or more of an Fc region from an IgG1, IgG2, IgG3, IgG4, IgA, IgM, IgE, or IgD. In some aspects, the antibody is an IgG1 antibody. In some aspects, the antibody is an IgG2 antibody. In some aspects, the antibody is an IgG3 antibody. In some aspects, the antibody is an IgG4 antibody.

In some aspects, the bispecific binding protein (e.g., AZD7789) comprises a variant Fc region. Fc region engineering is widely used in the art to extend the half-life of therapeutic antibodies and protect from degradation in vivo. In some aspects, the Fc region of an IgG antibody or antigen-binding fragment can be modified in order to increase the affinity of the IgG molecule for the Fc Receptor-neonate (FcRn), which mediates IgG catabolism and protects IgG molecules from degradation.

In some aspects, the variant Fc region of the bispecific binding protein comprises at least one substitution selected from 221K, 221Y, 225E, 225K, 225W, 228P, 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235E, 235F, 236E, 237L, 237M, 237P, 239D, 239E, 239N, 239Q, 239F, 239T, 239H, 239Y, 2401, 240A, 240T, 240M, 241W, 241L, 241Y, 241E, 241R, 243W, 243L 243Y, 243R, 243Q, 244H, 245A, 247L, 247V, 247G, 250E, 250Q, 251F, 252L, 252Y, 254S, 254T, 255L, 256E, 256F, 256M, 257C, 257M, 257N, 2621, 262A, 262T, 262E, 2631, 263A, 263T, 263M, 264L, 2641, 264W, 264T, 264R, 264F, 264M, 264Y, 264E, 265A, 265G, 265N, 265Q, 265Y, 265F, 265V, 2651, 265L, 265H, 265T, 2661, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 2961, 296H, 296G, 297S, 297D, 297E, 298A, 298H, 2981, 298T, 298F, 2991, 299L, 299A, 299S, 299V, 299H, 299F, 299E, 3051, 308F, 313F, 316D, 318A, 318S, 320A, 320S, 322A, 322S, 325Q, 325L, 3251, 325D, 325E, 325A, 325T, 325V, 325H, 326A, 326D, 326E, 326G, 326M, 326V, 327G, 327W, 327N, 327L, 328S, 328M, 328D, 328E, 328N, 328Q, 328F, 3281, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 333A, 333D, 333G, 333Q, 333S, 333V, 334A, 334E, 334H, 334L, 334M, 334Q, 334V, 334Y, 339T, 370E, 370N, 378D, 392T, 396L, 416G, 419H, 421K, 428L, 428F, 433K, 433L, 434A, 434W, 434Y, 436H, 440Y and 443W as numbered by the EU index as set forth in Kabat.

In some aspects, the variant Fc region comprises one or more modifications at positions selected from 428 and 434 as numbered by the EU index as set forth in Kabat. In some aspects, the variant Fc region comprises one or more amino acid substitutions at positions selected from 428 and 434 as numbered by the EU index as set forth in Kabat. In some aspects, the variant Fc region comprises one or more amino acid substitutions selected from 428L, 428F, 434A, 424F, 434W, and 434Y.

In some aspects, the variant Fc region of the bispecific binding protein comprises one or more amino acid substitutions at positions selected from 428 and 434 as numbered by the EU index as set forth in Kabat. In some aspects, the variant Fc region of the bispecific binding protein comprises one or more amino acid substitutions selected from 428L, 428F, 434A, 424F, 434W, and 434Y. In some aspects, the variant Fc region of the bispecific binding protein comprises a YTE mutation (M252Y/S254T/T256E).

In some aspects, the Fc variant antibody or binding fragment thereof has reduced antibody dependent cellular cytotoxicity (ADCC) when administered in vivo. In some aspects, the Fc variant antibody or binding fragment thereof has reduced ADCC compared to an antibody or binding variant thereof that contains a wild-type Fc region. In some aspects, the Fc variant antibody or binding fragment thereof does not trigger ADCC when administered in vivo. In some aspects, the Fc variant antibody or binding fragment thereof causes reduced ADCC when administered in vivo. In some aspects, the Fc variant antibody or binding fragment thereof having reduced ADCC activity or no ADCC activity comprises the L234F/L235E/P331S triple mutation (TM) in the variant Fc region.

In some aspects, the antibody or binding fragment thereof having reduced CDC activity has reduced toxicity when administered to a subject. In some aspects, the antibody or binding fragment thereof having reduced ADCC activity has reduced toxicity when administered to a subject.

In some aspects, the Fc region of the bispecific binding protein is aglycosylated. In some aspects, the Fc region of the bispecific binding protein is deglycosylated. In some aspects, the Fc region of the bispecific binding protein has reduced fucosylation or is afucosylated.

In some aspects, the bispecific binding protein comprises a kappa light chain constant region. In some aspects, the bispecific binding protein comprises a lambda light chain constant region.

In some aspects, the bispecific binding protein is an antibody. In some aspects, the antibody is an IgG antibody. In some aspects, the antibody is an IgG1 antibody. In some aspects, the antibody is an IgG2 antibody. In some aspects, the antibody is an IgG3 antibody. In some aspects, the antibody is an IgG4 antibody. In some aspects, the antibody is humanized.

In some aspects, the disclosure provides methods of inducing an immune response in a subject as well as methods for treating or preventing NSCLC or cHL in a subject by administering the proteins, nucleic acid molecules and/or compositions to the subject.

In some aspects, provided herein is a method of inducing an immune response in a subject comprising administering to the subject a bispecific protein (e.g., AZD7789) as described herein. In one aspect, provided herein is a method of inducing an immune response in a subject comprising administering to the subject a nucleic acid as described herein. In one aspect, provided herein is a method of inducing an immune response in a subject comprising administering to the subject a pharmaceutical composition as described herein.

In one aspect, provided herein is a bispecific protein (e.g., AZD7789) as defined herein for use in therapy. In one aspect, provided herein is a bispecific protein as defined herein for use in the treatment of NSCLC or cHL.

In one aspect, provided herein is the use of a bispecific protein as defined herein (e.g., AZD 7789) in the manufacture of a medicament for the treatment of NSCLC or cHL.

In one aspect, provided herein is a nucleic acid as defined herein for use in therapy. In one aspect, provided herein is a nucleic acid as defined herein for use in the treatment of NSCLC or cHL.

In one aspect, provided herein is the use of a nucleic acid as defined herein in the manufacture of a medicament for the treatment of NSCLC or cHL.

In some aspects, the binding proteins disclosed herein can be formulated with a pharmaceutically acceptable carrier, excipient, or stabilizer, as pharmaceutical compositions. In certain aspects, such pharmaceutical compositions are suitable for administration to a human or non-human animal intravenously. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which can be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

In some aspects, the amount of bispecific binding protein (e.g., AZD7789) administered NSCLC or cHL patient is about 50 mg to about 2000 mg. In some aspects, the amount of bispecific binding protein (e.g., AZD7789) administered to NSCLC or cHL patient is about 70 mg to about 1500 mg. In some aspects, the amount of bispecific binding protein (e.g., AZD7789) administered to NSCLC or cHL patient is about 100 mg to about 1400 mg. In some aspects, the amount of bispecific binding protein (e.g., AZD7789) administered to NSCLC or cHL patient is about 200 mg to about 1250 mg. In some aspects, the amount of bispecific binding protein (e.g., AZD7789) administered to NSCLC or cHL patient is about 500 mg to about 1000 mg. In some aspects, the amount of bispecific binding protein (e.g., AZD7789) administered to NSCLC or cHL patient is about 600 mg to about 900 mg. In some aspects, the amount of bispecific binding protein administered to NSCLC or cHL patient is about 700 mg to about 800 mg.

In some aspects, the amount of bispecific binding protein (e.g., AZD7789) administered to treat NSCLC or cHL is about 50 mg, about 100 mg, about 150 mg, about 200mg, about 225 mg, about 250 mg, about 300 mg, about 350 mg, about 400 mg, about 450 mg, about 500 mg, about 550 mg, about 600 mg, about 650 mg, about 700 mg, about 725 mg, about 750 mg, about 775 mg, about 800 mg, about 850 mg, about 900 mg, about 950 mg, about 1000mg, about 1050 mg, about 1100 mg, about 1150 mg, about 1200 mg, about 1250 mg, about 1300mg, about 1350 mg, about 1440 mg, about 1450 mg, about 1500 mg, about 1550 mg, about 1600mg, about 1650 mg, about 1700 mg, about 1750 mg, about 1800 mg, about 1850 mg, about 1900mg, about 1950 mg, or about 2000 mg.

In some aspects, the amount of bispecific binding protein (e.g., AZD7789) administered to treat NSCLC or cHL is about 70 mg, about 150 mg, about 210 mg, about 225 mg, about 450 mg, about 750 mg, about 800 mg, about 850 mg, about 900 mg, about 950 mg, about 1000 mg, about 1250 mg, or about 1500 mg.

In some aspects, the amount of bispecific binding protein (e.g., AZD7789) administered to treat NSCLC or cHL is about 750 mg. In some aspects, the amount of bispecific binding protein (e.g., AZD7789) administered is about 1500 mg.

In some aspects, the amount of bispecific binding protein (e.g., AZD7789) administered to treat NSCLC or cHL is 70 mg, 150 mg, 210 mg, 225 mg, 450 mg, 750 mg, 800mg, 850 mg, 900 mg, 950 mg, 1000 mg, 1250 mg, or 1500 mg.

In some aspects, the amount of bispecific binding protein (e.g., AZD7789) administered to treat NSCLC or cHL is 750 mg. In some aspects, the amount of bispecific binding protein administered to treat NSCLC or cHL is 1500 mg.

In some aspects, the bispecific binding protein (e.g., AZD7789) is administered to treat NSCLC or cHL once per treatment cycle. In some aspects, the bispecific binding protein (e.g., AZD7789) is administered to treat NSCLC or cHL twice per treatment cycle. In some aspects, the bispecific binding protein (e.g., AZD7789) is administered to treat NSCLC or cHL three times per treatment cycle.

In some aspects, the treatment cycle is about 7 days, 14 days, 21 days, 28 days, or 35days. In some aspects, the treatment cycle is about 7 days. In some aspects, the treatment cycle is about 21 days.

In some aspects, the treatment cycle is 21 days and the bispecific protein is administered to treat NSCLC or cHL once per treatment cycle, i.e., Q3W dosing.

In some aspects, the treatment cycle is repeated for up to about 10 to about 100 cycles. In some aspects, the treatment cycle is repeated for up to about 20 to about 50 cycles. In some aspects, the treatment cycle is repeated for up to about 30 to about 40 cycles. In some aspects, the treatment cycle is repeated for up to 10, 15, 20, 25, 30, 35, 40, 45 or 50 cycles. In some aspects, the treatment cycle is repeated for up to 35 cycles.

In some aspects, the bispecific binding protein is administered to treat NSCLC or cHL to the subject as a monotherapy or a combinational therapy. In some aspects, the bispecific binding protein is administered to treat NSCLC or cHL to the subject as a monotherapy.

In one aspect, the method comprises administering to the subject the binding protein (e.g., AZD7789) to treat NSCLC or cHL disclosed herein in combination with an additional standard of care (SoC) anti-cancer compound (e.g., chemotherapy). In some aspects, the SoC anti-cancer compound is pemetrexed, carboplatin, gemcitabine, cisplatin, paclitaxel or combinations thereof. In some aspects, the binding protein and additional anti-cancer SoC treatment are administered simultaneously. In some aspects, the binding protein and additional SoC anti-cancer treatment are not administered simultaneously but are administered during the same treatment cycle.

In some aspects, the bispecific binding protein is administered by an intravenous infusion (IV).

In some aspects, the subject has not received a prior line of systemic therapy. In some aspects, the subject is checkpoint inhibitor (CPI) naïve, meaning that that the subject has not previously been administered a CPI.

In some aspects, the subject has previously received a chemotherapy. In some aspects, the chemotherapy includes a platinum-based chemotherapy. In some aspects, the prior line of systematic therapy includes checkpoint inhibitor (CPI) therapy.

In some aspects, the subject has previously received a prior line of immuno-oncology (IO) therapy. In some aspects, the subject has received CPI. In some aspects, the subject has previously received an anti-PD1/PD-L1 IO therapy.

In some aspects, the anti-PD-1 therapy is an antibody selected from nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), cemiplimab (Regeneron; also known as REGN-2810; see WO 2015/112800), JS001 (TAIZHOU JUNSHI PHARMA; see Si-Yang Liu et al., J. Hematol. Oncol. 70:136 (2017)), BGB-A317 (Beigene; see WO 2015/35606 and US 2015/0079109), INCSHR 1210 (Jiangsu Hengrui Medicine; also known as SHR-1210; see WO 2015/085847; Si-Yang Liu et al, J Hematol. Oncol. 70:136 (2017)), TSR-042 (Tesaro Biopharmaceutical; also known as ANB011; see WO2014/179664), Pidilizumab (Medivation/CureTech; see U.S. Pat. No. 8,686,119 B2 or WO 2013/014668 A1); GLS-010 (Wuxi/Harbin Gloria Pharmaceuticals; also known as WBP3055; see Si-Yang Liu et al, J. Hematol. Oncol. 70:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), and IBI308 (Innovent; see WO 2017/024465, WO 2017/025016, WO 2017/132825, and WO 2017/133540). In some aspects the anti-PD-1 therapy is the PD-1 antagonist AMP-224, which is a recombinant fusion protein comprised of the extracellular domain of the PD-1 ligand programmed cell death ligand 2 (PD-L2) and the Fc region of human IgG. AMP-224 is discussed in U.S. Publ. No. 2013/0017199. The contents of each of these references are incorporated by reference herein in their entirety.

In some aspects, the anti-PD-L1 therapy is an antibody selected from BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 3 1 (suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), and CK-301 (Checkpoint Therapeutics; see Gorelik et al., AACR: Abstract 4606 (April 2016)), The contents of each of these references are incorporated by reference herein in their entirety.

In some aspects of the methods disclosed herein, the subject (e.g., NSCLC or cHL patient) has immune-oncology (IO) acquired resistance. In some aspects, the subject has acquired resistance to an anti-PD1 and/or an anti-PD-L1 IO therapy. In some aspects, the subject with IO acquired resistance has a radiologically documented tumor progression or clinical deterioration following initial treatment with an anti-PD-1/PD-L1 therapy for a minimum of 3-6 months, as monotherapy or in combination with chemotherapy, and had signs of initial clinical benefit, i.e. disease stabilization or regression.

In some aspects of the methods disclosed herein, IO acquired resistance is defined as:
(i) Exposure of less than 6 months to anti-PD-1/PD-L1 monotherapy with initial best overall response (BOR) of partial regression or complete regression followed by disease progression during treatment or disease progression less than or equal to 12 weeks after anti-PD-1/PD-L1 treatment discontinuation; or
(ii) Exposure of greater than or equal to 6 months to anti-PD-1/PD-L1 therapy alone or in combination with chemotherapy with BOR of disease stabilization, partial regression, or complete regression followed by disease progression during treatment or disease progression less than or equal to 12 weeks after anti-PD-1/PD-L1 treatment discontinuation.

In some aspects of the methods disclosed herein, the IO acquired resistance is defined as exposure of greater than or equal to 6 months to anti-PD-1/PD-L1 therapy alone or in combination with chemotherapy; a best overall response (BOR) of disease stabilization, partial regression, or complete regression followed by disease progression during treatment or disease progression less than or equal to 12 weeks after anti-PD-1/PD-L1 treatment discontinuation.

In some aspects, NSCLC or cHL comprises a NSCLC or cHL cell which expresses PD-L1.

In some aspects, the subject has documented Stage III NSCLC which is not amenable to curative surgery or radiation.

In some aspects, the NSCLC is advanced or metastatic. In some aspects, the subject has Stage IV non-small cell lung carcinoma (NSCLC). In some aspects, the NSCLC is squamous or non-squamous NSCLC.

In aspects where is the subject has NSCLC, the subject has a PD-L1 tumor proportion score of greater than or equal to 1%. In some aspects where is the subject has NSCLC, the subject has a PD-L1 tumor proportion score of greater than or equal to 50%. In some aspects, the PD-L1 tumor proportion score can be determined using the Ventana PD-L1 SP263 Assay.

In some aspects, the cHL is relapsed or refractory.

### Pharmaceutical Compositions

In some aspects, the present disclosure further provides a pharmaceutical composition comprising a bispecific binding protein that specifically binds to PD-1 and TIM-3 in an amount from about 70 mg to about 1500 mg. In some aspects, the bispecific binding protein comprising: a) a first binding domain that specifically binds to PD-1, wherein the first binding domain comprises a heavy chain variable domain comprising a HCDR1 having the amino acid sequence of SEQ ID NO: 4, a HCDR2 having the amino acid sequence of SEQ ID NO: 5, and a HCDR3 having the amino acid sequence of SEQ ID NO: 6, and a light chain variable domain comprising a LCDR1 having the amino acid sequence of SEQ ID NO: 10, a LCDR2 having the amino acid sequence of SEQ ID NO: 11 and a LCDR3 having the amino acid sequence of SEQ ID NO: 12; and b) a second binding domain that specifically binds to TIM-3, wherein the second binding domain comprises a heavy chain variable domain comprising a HCDR1 having the amino acid sequence of SEQ ID NO: 1, a HCDR2 having the amino acid sequence of SEQ ID NO: 2, and a HCDR3 having the amino acid sequence of SEQ ID NO: 3, and a light chain variable domain comprising a LCDR1 having the amino acid sequence of SEQ ID NO: 7, a LCDR2 having the amino acid sequence of SEQ ID NO: 8, and a LCDR3 having the amino acid sequence of SEQ ID NO: 9.

In some aspects, the amount of bispecific binding protein administered to treat NSCLC or cHL is about 50 mg to about 2000 mg. In some aspects, the amount of bispecific binding protein administered to treat NSCLC or cHL is about 70 mg to about 1500 mg. In some aspects, the amount of bispecific binding protein administered to treat NSCLC or cHL is about 100 mg to about 1400 mg. In some aspects, the amount of bispecific binding protein administered to treat NSCLC or cHL is about 200 mg to about 1250 mg. In some aspects, the amount of bispecific binding protein administered to treat NSCLC or cHL is about 500 mg to about 1000 mg. In some aspects, the amount of bispecific binding protein administered to treat NSCLC or cHL is about 600 mg to about 900 mg. In some aspects, the amount of bispecific binding protein administered to treat NSCLC or cHL is about 700 mg to about 800 mg.

In some aspects, the amount of bispecific binding protein (e.g., AZD7789) administered to treat NSCLC or cHL is about 50 mg, about 100 mg, about 150 mg, about 200 mg, about 225 mg, about 250 mg, about 300 mg, about 350 mg, about 400 mg, about 450 mg, about 500 mg, about 550 mg, about 600 mg, about 650 mg, about 700 mg, about 725 mg, about 750 mg, about 775 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1440 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some aspects, the amount of bispecific binding protein administered to treat NSCLC or cHL is about 70 mg, about 150 mg, about 210 mg, about 225 mg, about 450 mg, about 750 mg, about 800 mg, about 850 mg, about 900 mg, about 950 mg, about 1000 mg, about 1250 mg, or about 1500 mg.

In some aspects, the amount of bispecific binding protein administered to treat NSCLC or cHL is about 750 mg. In some aspects, the amount of bispecific binding protein administered to treat NSCLC or cHL is about 1500 mg.

In some aspects, the amount of bispecific binding protein administered to treat NSCLC or cHL is 70 mg, 150 mg, 210 mg, 225 mg, 450 mg, 750 mg, 800 mg, 850 mg, 900 mg, 950 mg, 1000 mg, 1250 mg, or 1500 mg.

In some aspects, the amount of bispecific binding protein administered to treat NSCLC or cHL is 750 mg. In some aspects, the amount of bispecific binding protein administered to treat NSCLC or cHL is 1500 mg.

In some aspects, the first binding domain of the bispecific binding protein that specifically binds to PD-1 comprises a heavy chain variable domain having the amino acid sequence of SEQ ID NO: 19 and a light chain variable domain having the amino acid sequence of SEQ ID NO:21. In some aspects, the first binding domain of the bispecific binding protein that specifically binds to PD-1 comprises a heavy chain variable domain having an amino acid sequence that is at least 90% identical to the amino acid sequence of SEQ ID NO: 19 and a light chain variable domain having an amino acid sequence that is at least 90% identical to the amino acid sequence of SEQ ID NO:21.

In some aspects, the second binding domain of the bispecific binding protein that specifically binds to TIM-3 comprises a heavy chain variable domain having the amino acid sequence of SEQ ID NO: 14 and a light chain variable domain having the amino acid sequence of SEQ ID NO:17. In some aspects, the second binding domain of the bispecific binding protein that specifically binds to TIM-3 comprises a heavy chain variable domain having an amino acid sequence that is at least 90% identical to the amino acid sequence of SEQ ID NO: 14 and a light chain variable domain having an amino acid sequence that is at least 90% identical to the amino acid sequence of SEQ ID NO:17.

In some aspects, the pharmaceutical compositions disclosed herein can be formulated with a pharmaceutically acceptable carrier, excipient, or stabilizer. In certain aspects, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which can be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

The present disclosure further provides a pharmaceutical composition as defined above, for use in treating NSCLC or cHL.

In some aspects of the composition for use, the cancer is NSCLC or cHL.

In some aspects, the subject has documented Stage III NSCLC which is not amenable to curative surgery or radiation.

In some aspects, the subject to be treated has is non-small cell lung cancer (NSCLC). In some aspects, the NSCLC is advanced or metastatic. In some aspects, the subject has Stage IV non-small cell lung carcinoma (NSCLC). In some aspects, the NSCLC is squamous or non-squamous NSCLC.

In some aspects where the subject to be treated has NSCLC, the subject has a PD-L1 tumor proportion score of greater than or equal to 1%. In some aspects where the subject to be treat has NSCLC, the subject has a PD-L1 tumor proportion score of greater than or equal to 50%.

In some aspects of the composition for use, the NSCLC or cHL has not been previously treated. In some aspects, the NSCLC or cHL is checkpoint inhibitor (CPI) naïve.

In some aspects of the composition for use, the NSCLC or cHL has previously been treated with a chemotherapy. In some aspects, the chemotherapy includes a platinum-based chemotherapy. In some aspects, the NSCLC or cHL has been treated with a CPI.

In some aspects of the composition for use, the NSCLC or cHL has been previously treated with an immuno-oncology (IO) therapy. In some aspects, the NSCLC or cHL has been previously treated with an anti-PD1/PD-L1 IO therapy. Examples of anti-PD1/PD-L1 IO therapies are provided elsewhere herein.

In some aspects of composition for use, the NSCLC or cHL has immune-oncology (IO) acquired resistance. In some aspects, the NSCLC or cHL has acquired resistance to an anti-PD1 and/or an anti-PD-L1 IO therapy.

### Kits

In some aspects, the present disclosure further provides a kit comprising any of the pharmaceutical compositions described above. In some aspects, the kit comprises instructions for administering the pharmaceutical composition. In some aspects, the kit comprises an additional anti-cancer agent as described herein.

### Bispecific Binding Molecule Sequences

Aspects of the disclosure include bispecific binding proteins in DuetMab format that bind PD-1 and TIM-3 created using the sequences in Table 1 below. CDRs in Table 1 are determined based on the system in Kabat.

TABLE 1
Sequences for PD-1/TIM-3 Bispecific
Binding Molecules

| Description | Sequence |
|---|---|
| AZD 7789 | SYAMS |
| TIM-3 | (SEQ ID NO: 1) |
| VH CDR1 | |
| AZD 7789 | AISGSGGSTYYADSVKG |
| TIM-3 | (SEQ ID NO: 2) |
| VH CDR2 | |
| AZD 7789 | GSYGTYYGNYFEY |
| TIM-3 | (SEQ ID NO: 3) |
| VH CDR3 | |
| AZD 7789 | DYGMH |
| PD-1 | (SEQ ID NO: 4) |
| VH CDR1 | |
| AZD 7789 | YISSGSYTIYSADSVKG |
| PD-1 | (SEQ ID NO: 5) |
| VH CDR2 | |
| AZD 7789 | RAPNSFYEYYFDY |
| PD-1 | (SEQ ID NO: 6) |
| VH CDR3 | |
| AZD 7789 | GGDNIGGKSVH |
| TIM-3 | (SEQ ID NO: 7) |
| VL CDR1 | |
| AZD 7789 | YDSDRPS |
| TIM-3 | (SEQ ID NO: 8) |
| VL CDR2 | |
| AZD 7789 | QVLDRRSDHFL |
| TIM-3 | (SEQ ID NO: 9) |
| VL CDR3 | |
| AZD 7789 | SASSKHTNLYWSRHMYWY |
| PD-1 | (SEQ ID NO: 10) |
| VL CDR1 | |
| AZD 7789 | LTSNRAT |
| PD-1 | (SEQ ID NO: 11) |
| VL CDR2 | |
| AZD 7789 | QQWSSNP |
| PD-1 | (SEQ ID NO: 12) |
| VL CDR3 | |
| TIM-3 (#62) | QVLDRRSDHWL |
| VL CDR3 | (SEQ ID NO: 13) |
| AZD 7789 | EVQLLESGGGLVQPGGSLRLSCAASGFTFS |
| TIM-3 | SYAMSWVRQAPGKGLEWVSAISGSGGSTYY |
| VH | ADSVKGRFTISRDNSKNTLYLQMNSLRAED |
| | TAVYYCARGSYGTYYGNYFEYWGQGTLVTV |
| | SS |
| | (SEQ ID NO: 14) |
| AZD 7789 | EVQLLESGGGLVQPGGSLRLSCAASGFTFS |
| TIM-3 | SYAMSWVRQAPGKGLEWVSAISGSGGSTYY |
| HC | ADSVKGRFTISRDNSKNTLYLQMNSLRAED |
| | TAVYYCARGSYGTYYGNYFEYWGQGTLVTV |
| | SSASTKGPSVCPLAPSSKSTSGGTAALGCL |
| | VKDYFPEPVTVSWNSGALTSGVHTFPAVLQ |
| | SSGLYSLSSVVTVPSSSLGTQTYICNVNHK |
| | PSNTKVDKRVEPKSVDKTHTCPPCPAPEFE |
| | GGPSVFLFPPKPKDTLMISRTPEVTCVVVD |
| | VSHEDPEVKFNWYVDGVEVHNAKTKPREEQ |
| | YNSTYRVVSVLTVLHQDWLNGKEYKCKVSN |
| | KALPASIEKTISKAKGQPREPQVYTLPPCR |
| | EEMTKNQVSLWCLVKGFYPSDIAVEWESNG |
| | QPENNYKTTPPVLDSDGSFFLYSKLTVDKS |
| | RWQQGNVFSCSVMHEALHNHYTQKSLSLSP |
| | GK |
| | (SEQ ID NO: 15) |
| TIM3 (#62) | QTVLTQPPSVSVAPGKTASISCGGDNIGGK |
| Variable | SVHWYQQKPGQAPVLVIYYDSDRPSGIPQR |
| Light VL | FSGSNSGNTATLTIHRVEAGDEADYYCQVL |
| | DRRSDHWLFGGGTKLTVL |
| | (SEQ ID NO: 16) |
| AZD 7789 | SYVLTQPPSVSVAPGKTARITCGGDNIGGK |
| TIM-3 | SVHWYQQKPGQAPVLVIYYDSDRPSGIPER |
| VL | FSGSNSGNTATLTISRVEAGDEADYYCQVL |
| | DRRSDHFLFGGGTKLTVL |
| | (SEQ ID NO: 17) |
| AZD 7789 | SYVLTQPPSVSVAPGKTARITCGGDNIGGK |
| TIM-3 | SVHWYQQKPGQAPVLVIYYDSDRPSGIPER |
| LC | FSGSNSGNTATLTISRVEAGDEADYYCQVL |
| | DRRSDHFLFGGGTKLTVLGQPKAAPSVTLF |
| | PPCSEELQANKATLVCLISDFYPGAVTVAW |
| | KADSSPVKAGVETTTPSKQSNNKYAASSYL |
| | SLTPEQWKSHRSYSCQVTHEGSTVEKTVAP |
| | TEVS |
| | (SEQ ID NO: 18) |
| AZD | EVQLVESGGGLVQPGGSLRLSCAASGFTFS |
| 7789 | DYGMHWVRQAPGKGLEWVAYISSGSYTIYS |
| PD-1 | ADSVKGRFTISRDNAKNSLYLQMNSLRAED |
| VH | TAVYYCARRAPNSFYEYYFDYWGQGT |
| | TVTVSS |
| | (SEQ ID NO: 19) |
| AZD 7789 | EVQLVESGGGLVQPGGSLRLSCAASGFTFS |
| PD-1 | DYGMHWVRQAPGKGLEWVAYISSGSYTIYS |
| HC | ADSVKGRFTISRDNAKNSLYLQMNSLRAED |
| | TAVYYCARRAPNSFYEYYFDYWGQGTTVTV |
| | SSASTKGPSVFPLAPSSKSTSGGTAALGCL |
| | VKDYFPEPVTVSWNSGALTSGVHTFPAVLQ |
| | SSGLYSLSSVVTVPSSSLGTQTYICNVNHK |
| | PSNTKVDKRVEPKSCDKTHTCPPCPAPEFE |
| | GGPSVFLFPPKPKDTLMISRTPEVTCVVVD |
| | VSHEDPEVKFNWYVDGVEVHNAKTKPREEQ |
| | YNSTYRVVSVLTVLHQDWLNGKEYKCKVSN |
| | KALPASIEKTISKAKGQPREPQVCTLPPSR |
| | EEMTKNQVSLSCAVKGFYPSDIAVEWESNG |
| | QPENNYKTTPPVLDSDGSFFLVSKLTVDKS |
| | RWQQGNVFSCSVMHEALHNHYTQKSLSLSP |
| | GK |
| | (SEQ ID NO: 20) |
| AZD 7789 | QIVLTQSPATLSLSPGERATLSCSASSKHT |
| PD-1 | NLYWSRHMYWYQQKPGQAPRLLIYLTSNRA |
| VL | TGIPARFSGSGSGTDFTLTISSLEPEDFAV |
| | YYCQQWSSNPFTFGQGTKLEIK |
| | (SEQ ID NO: 21) |
| AZD 7789 | QIVLTQSPATLSLSPGERATLSCSASSKHT |
| PD-1 | NLYWSRHMYWYQQKPGQAPRLLIYLTSNRA |
| LC | TGIPARFSGSGSGTDFTLTISSLEPEDFAV |
| | YYCQQWSSNPFTFGQGTKLEIKRTVAAPSV |
| | FIFPPSDEQLKSGTASVVCLLNNFYPREAK |
| | VQWKVDNALQSGNSQESVTEQDSKDSTYSL |
| | SSTLTLSKADYEKHKVYACEVTHQGLSSPV |
| | TKSFNRGEC |
| | (SEQ ID NO: 22) |
| TIM-3 Heavy | EVQLVESGGGLVQPGGSLRLSCAASGFTFS |
| Chain | DYGMHWVRQAPGKGLEWVAYISSGSYTIYS |
| | ADSVKGRFTISRDNAKNSLYLQMNSLRAED |
| | TAVYYCARRAPNSFYEYYFDYWGQGTTVTV |
| | SSASTKGPSVFPLAPSSKSTSGGTAALGCL |
| | VKDYFPEPVTVSWNSGALTSGVHTFPAVLQ |
| | SSGLYSLSSVVTVPSSSLGTQTYICNVNHK |
| | PSNTKVDKRVEPKSCDKTHTCPPCPAPEFE |
| | GGPSVFLFPPKPKDTLMISRTPEVTCVWDV |
| | SHEDPEVKFNWYVDGVEVHNAKTKPREEQY |
| | NSTYRWSVLTVLHQDWLNGKEYKCKVSNKA |
| | LPASIEKTISKAKGQPREPQVYTLPPSREE |
| | MTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPGK |
| | GGGGSGGGGSEVQLLESGGGLVQPGGSLRL |
| | SCAASGFTFSSYAMSWVRQAPGKCLEWVSA |
| | ISGSGGSTYYADSVKGRFTISRDNSKNTLY |
| | LQMNSLRAEDTAVYYCARGSYGTYYGNYFE |
| | YWGQGTLVTVSSGGGGSGGGGSGGGGSGGG |
| | GSSYVLTQPPSVSVAPGKTARITCGGDNIG |
| | GKSVHWYQQKPGQAPVLVIYYDSDRPSGIP |
| | ERFSGSNSGNTATLTISRVEAGDEADYYCQ |
| | VLDRRSDHFLFGCGTKLTVL |
| | (SEQ ID NO: 23) |
| TIM-3 Light | QIVLTQSPATLSLSPGERATLSCSASSKHT |
| Chain | NLYWSRHMYWYQQKPGQAPRLLIYLTSNRA |
| Variable | TGIPARFSGSGSGTDFTLTISSLEPEDFAV |
| Region | YYCQQWSSNPFTFGQGTKLEIKRTVAAPSV |
| | FIFPPSDEQLKSGTASWCLLNNFYPREAKV |
| | QWKVDNALQSGNSQESVTEQDSKDSTYSLS |
| | STLTLSKADYEKHKVYACEVTHQGLSSPVT |
| | KSFNRGEC |
| | (SEQ ID NO: 24) |
| TIM-3 Heavy | EVQLVESGGGLVQPGGSLRLSCAASGFTFS |
| Chain | DYGMHWVRQAPGKGLEWVAYISSGSYTIYS |
| | ADSVKGRFTISRDNAKNSLYLQMNSLRAED |
| | TAVYYCARRAPNSFYEYYFDYWGQGTTVTV |
| | SSASTKGPSVFPLAPSSKSTSGGTAALGCL |
| | VKDYFPEPVTVSWNSGALTSGVHTFPAVLQ |
| | SSGLYSLSSVVTVPSSSLGTQTYICNVNHK |
| | PSNTKVDKRVEPKSCDKTHTCPPCPAPEFE |
| | GGPSVFLFPPKPKDTLMISRTPEVTCVWDV |
| | SHEDPEVKFNWYVDGVEVHNAKTKPREEQY |
| | NSTYRWSVLTVLHQDWLNGKEYKCKVSNKA |
| | LPASIEKTISKAKGQPREPQVYTLPPSREE |
| | MTKNQVSLTCLVKGFYPSDIAVEWESNGGG |
| | GSGGGGSEVQLLESGGGLVQPGGSLRLSCA |
| | ASGFTFSSYAMSWVRQAPGKCLEWVSAISG |
| | SGGSTYYADSVKGRFTISRDNSKNTLYLQM |
| | NSLRAEDTAVYYCARGSYGTYYGNYFEYWG |
| | QGTLVTVSSGGGGSGGGGSGGGGSGGGGSS |
| | YVLTQPPSVSVAPGKTARITCGGDNIGGKS |
| | VHWYQQKPGQAPVLVIYYDSDRPSGIPERF |
| | SGSNSGNTATLTISRVEAGDEADYYCQVLD |
| | RRSDHFLFGCGTKLTVLGGGGSGGGGSGQP |
| | ENNYKTTPPVLDSDGSFFLYSKLTVDKSRW |
| | QQGNVFSCSVMHEALHNHYTQKSLSLSPGK |
| | (SEQ ID NO: 25) |
| TIM-3 Light | QIVLTQSPATLSLSPGERATLSCSASSKHT |
| Chain | NLYWSRHMYWYQQKPGQAPRLLIYLTSNRA |
| | TGIPARFSGSGSGTDFTLTISSLEPEDFAV |
| | YYCQQWSSNPFTFGQGTKLEIKRTVAAPSV |
| | FIFPPSDEQLKSGTASWCLLNNFYPREAKV |
| | QWKVDNALQSGNSQESVTEQDSKDSTYSLS |
| | STLTLSKADYEKHKVYACEVTHQGLSSPVT |
| | KSFNRGEC |
| | (SEQ ID NO: 26) |
| TIM3 (#62) | EVQLLESGGGLVQPGGSLRLSCAASGFTFS |
| Variable | SYAMSWVRQAPGKGLEWVSAISGSGGSTYY |
| Heavy VH | ADSVKGRFTISRDNSKNTLYLQMNSLRAED |
| | TAVYYCARGSYGTYYGNYFEYWGRGTLVTV |
| | SS |
| | (SEQ ID NO: 27) |
| Amino acid | MQIPQAPWPVVWAVLQLGWRPGWFLDSPDR |
| sequence of | PWNPPTFSPALLVVTEGDNATFTCSFSNTS |
| human PD-1 | ESFVLNWYRMSPSNQTDKLAAFPEDRSQPG |
| protein | QDCRFRVTQLPNGRDFHMSVVRARRNDSGT |
| | YLCGAISLAPKAQIKESLRAELRVTERRAE |
| | VPTAHPSPSPRPAGQFQTLVVGVVGGLLGS |
| | LVLLVWVLAVICSRAARGTIGARRTGQPLK |
| | EDPSAVPVFSVDYGELDFQWREKTPEPPVP |
| | CVPEQTEYATIVFPSGMGTSSPARRGSADG |
| | PRSAQPLRPEDGHCSWPL |
| | (SEQ ID NO: 28) |
| Human TIM-3 | SEVEYRAEVGQNAYLPCFYTPAAPGNLVPV |
| IgV | CWGKGACPVFECGNVVLRTDERDVNYWTSR |
| domain | YWLNGDFRKGDVSLTIENVTLADSGIYCCR |
| | IQIPGIMNDEKFNLKLVIK |
| | (SEQ ID NO: 29) |
| Human TIM-3 | MFSHLPFDCVLLLLLLLLTRSSEVEYRAEV |
| protein | GQNAYLPCFYTPAAPGNLVPVCWGKGACPV |
| | FECGNVVLRTDERDVNYWTSRYWLNGDFRK |
| | GDVSLTIENVTLADSGIYCCRIQIPGIMND |
| | EKFNLKLVIKPAKVTPAPTRQRDFTAAFPR |
| | MLTTRGHGPAETQTLGSLPDINLTQISTLA |
| | NELRDSRLANDLRDSGATIRIGIYIGAGIC |
| | AGLALALIFGALIFKWYSHSKEKIQNLSLI |
| | SLANLPPSGLANAVAEGIRSEENIYTIEEN |
| | VYEVEEPNEYYCYVSSRQQPSQPLGCRFAM |
| | P |
| | (SEQ ID NO: 30) |

The use of an anti-TIM-3/anti-PD-1 bispecific antibody is an innovative approach for targeting both TIM-3/PD-1 receptors at the same time. This approach has several potential advantages as compared with the co-administration of separate anti-TIM-3 and anti-PD-1/PD-L1 antibodies. Along with the case of drug delivery (a single administration replacing 2 administrations), there is a certainty of equal biodistribution of a single molecule targeting both receptors. Furthermore, the design of a bispecific antibody allows two potentially different modes of action, one targeting both receptors in close proximity on the same cell (cis effect) and another targeting the two receptors on two neighboring cells with the generation of a prolonged immune synapse simultaneously with checkpoint inhibition (trans effects). Dual targeting of PD-1 and TIM-3 has the potential to reinvigorate an immune response in participants who develop progression on prior PD-1/PD-L1 monotherapies and also can lead to more durable responses in participants who have not been previously treated with PD-1/PD-L1 checkpoint inhibitors, thereby resulting in a clinical benefit.

All references cited herein, including patents, patent applications, papers, textbooks and the like, and the references cited therein, to the extent that they are not already, are hereby incorporated herein by reference in their entirety.

Without limiting the disclosure, a number of aspects of the disclosure are described herein for purpose of illustration.

### EXAMPLES

The Examples that follow are illustrative of specific aspects of the disclosure, and various uses thereof. They are set forth for explanatory purposes only and should not be construed as limiting the scope of the disclosure in any way.

AZD7789 is provided for treatment in the Examples. AZD7789 is a monovalent bispecific humanized IgG1 with an engineered Fc domain to reduce Fc effector function (IgG1-triple mutation). AZD7789 specifically binds to human PD 1 and TIM-3. Programmed cell death protein 1 and TIM-3 are part of a complex system of cell surface receptors that, when bound to their cognate ligands, provide co-inhibitory signals to T cells to modulate their activity. Dual blockade of PD-1 and TIM-3 is expected to restore antitumor activity of dysfunctional T cells. AZD7789 is being developed for the treatment of subjects with advanced or metastatic NSCLC or Relapsed or Refractory Classical Hodgkin Lymphoma, with an initial focus on patients with primary or acquired resistance following prior treatment with PD-1/PD-L1 therapy.

### Example 1: NSCLC Study Design and Definitions

NSCLC is considered to be sensitive to immune checkpoint blockade, and for NSCLC, benefit has been seen for the addition of IO treatment regardless of PD-L1 expression. However, only a subset of patients respond to IO therapy and even those patients who initially respond to IO therapy may experience disease progression due to acquired resistance mediated by exhausted T cells overexpressing inhibitory receptors including PD-1 and TIM-3. In vitro and in vivo studies have demonstrated the ability of AZD7789 to engage TIM-3 and PD-1 and thereby promote enhanced immune mediated antitumor responses compared to targeting PD 1 alone. Therefore, targeting both PD-1 and TIM-3 with AZD7789 provides clinical benefit to subjects with advanced or metastatic NSCLC.

### Definitions—The Following Terms are Used in the Study Described in the Examples Below

Dose-limiting Toxicity (DLT). The DLTs will be evaluated during Part A (dose escalation). The DLT evaluation period will be 21 days from the first dose of AZD7789 on Cycle 1 Day 1. A DLT will be defined during Part A (dose escalation) as any ≥Grade 3 toxicity that occurs during the DLT evaluation period, with modifications or exceptions. Toxicity that is clearly attributable to the primary disease, other concomitant medications, disease-related processes under investigation, or another non-drug-related etiology (not related to treatment with study intervention) is excluded from this definition. All DLTs must be documented as AEs. All AEs will be graded according to NCI CTCAE v5.0.

Maximum Tolerated Dose (MTD). The MTD will be selected from all tried dose levels that have not been previously declared to be unsafe with a ‘DU’ (current dose is unacceptably toxic) decision according to the mTPI-2 algorithm. With this constraint, the MTD will be determined as the dose level with the DLT estimate closest to the target toxicity level of 30%.

In the case of dose levels with estimated toxicity of equal distance (tied dose levels) from the target toxicity of 30%, the following approach will be used (Ji Y, et al., A modified toxicity probability interval method for dose-finding trials, Clin Trials, 2010, 7(6):653-63) among all tied dose levels the highest dose level with target toxicity≤30% will be selected, unless all tied dose levels have estimated toxicity>30%, in which case the lowest dose level will be selected.

Efficacy Assessments. Tumor response will be assessed by RECIST v1.1 (Eisenhauer et al, 2009) according to the schedule (Q9W [±7 days] for 54 weeks then Q18W [±14 days] relative to the date of first dose [Cycle 1 Day 1] until disease progression or initiation of other anticancer therapy).

Tumor Evaluation. Tumor assessments will include physical examination and cross-sectional imaging using CT (preferred) or MRI scans. The CT scan of the chest, and CT or MRI scan of the abdomen, and pelvis will be performed at screening (with contrast, unless the subject has a documented intolerance). The preferred method of systemic disease assessment is CT with contrast; if CT with contrast is contraindicated, CT without contrast is preferred over MRI. The preferred method for brain imaging is MRI over CT (with contrast, unless the subject has a documented intolerance). The preferred method for bone imaging is bone scan. At screening, bone imaging is only applicable for subjects in whom there is a clinical suspicion of or confirmed bone metastases. Follow-up brain and bone imaging is required as clinically indicated if there are no metastases at baseline, and with every restaging scan if baseline brain or bone metastases are present. The same method should be used for all subsequent tumor assessments. Tumor assessments prior to baseline may also be requested, if available, provided these exams were performed within 6 months prior to the start of treatment. Circulating tumor deoxyribonucleic acid (ctDNA) and tumor antigen markers will be used, where available, as exploratory markers in the assessment of disease response.

Eastern Cooperative Oncology Group Performance Status. The ECOG performance status will be assessed at specified time points.

Adverse Events. An adverse event (AE) is the development of any untoward medical occurrence in a patient or clinical study subject administered a medicinal product and which does not necessarily have a causal relationship with this treatment. An AE can therefore be any unfavorable and unintended sign (e.g., an abnormal laboratory finding), symptom (for example nausea, chest pain), or disease temporally associated with the use of a medicinal product, whether or not considered related to the medicinal product.

The term AE is used to include both serious and non-serious AEs and can include a deterioration of a pre-existing medical occurrence. An AE may occur at any time, including run-in or washout periods, even if no Study treatment has been administered.

Serious Adverse Events. A serious adverse event (SAE) is an AE occurring during any study phase (i.e., run-in, treatment, washout, follow-up), that fulfils one or more of the following criteria: (1) Results in death; (2) Is immediately life-threatening; (3) Requires subject hospitalization or prolongation of existing hospitalization; (4) Results in persistent or significant disability or incapacity; (5) Is a congenital anomaly or birth defect; (6) Is an important medical event that may jeopardize the subject or may require medical treatment to prevent one of the outcomes listed above.

Disease Progression. Disease progression can be considered as a worsening of a subject's condition attributable to the disease for which the investigational product is being studied. It may be an increase in the severity of the disease under study and/or increases in the symptoms of the disease. The development of new or progression of existing metastasis to the primary cancer under study should be considered as disease progression and not an AE. Events that are unequivocally due to disease progression should not be reported as AEs during the study.

New Cancers. The development of a new cancer should be regarded as an SAE. New primary cancers are those that are not the primary reason for the administration of the study intervention and have been identified after the subject's inclusion in this study. They do not include metastases of the original cancer.

### Initial Dose Calculation

A population PK model was developed to describe the PK of AZD7789 in

cynomolgus monkeys. The half-life of AZD7789 in human was projected to be about 10 days; hence, a dosing interval of Q3W is proposed for this study.

A PK/pharmacodynamic model was developed to describe the dual target-mediated drug disposition (TMDD) of AZD7789 in humans. The linear PK parameters were set at typical human values for prior therapeutics. The saturable component of the TMDD model was assumed to be due to the binding of AZD7789 to the cell-surface PD-1 and TIM-3 as well as soluble TIM-3 in the periphery. For human simulations, the in vitro binding data including antibody affinity and antigen density as well as the antigen internalization data were incorporated in the model to predict the receptor occupancy of PD-1 and TIM-3 in the periphery following IV administration of AZD7789 at various dose levels. The starting dose of 2 mg AZD7789 corresponded to predicted levels of occupancy of PD-1 of approximately 21% and 77% of baseline at steady state Cₘᵢₙ and Cₘₐₓ of AZD7789, and TIM-3 occupancy of 5.8% and 47% of baseline at steady state Cₘᵢₙ and Cₘₐₓ of AZD7789.

### Study Design

This is a first-time-in-human (FTIH), multicenter, open-label, dose-escalation and dose-expansion study to evaluate the safety, tolerability, PK, pharmacodynamics, and antitumor activity of AZD7789 in adult subjects with advanced or metastatic non-small cell lung cancer (NSCLC) and other solid tumors. The study includes 2 parts: Part A Dose Escalation and Part B Dose Expansion. Initially, subjects with Stage IIIB to IV NSCLC will be enrolled in the study; additional tumor types may be explored and added in a future amendment to the clinical study protocol.

Part A Dose Escalation will evaluate approximately 8 dose levels of AZD7789 in subjects with Stage IIIB to IV NSCLC with PD-L1 expression<1% or ≥1% who have anti-PD-1/PD-L1 immune-oncology (IO) primary or acquired resistance in order to determine a MTD or OBD and a RP2D.

Dose escalation for the first 5 dose levels of AZD7789 (2, 7, 22.5, 75, and 225 mg) is planned to follow an accelerated titration design (ATD) consisting of 5 single-subject cohorts. Dose escalation for subsequent dose levels of AZD7789 (750, 1500, and 2000 mg) will follow the modified toxicity probability interval (mTPI-2) algorithm consisting of a minimum of 3 and a maximum of 12 subjects per dose level. If predefined safety criteria are met in an ATD cohort, dose escalation will switch to the mTPI-2 algorithm for all subsequent dose levels. Intermediate dose levels (50, 150, 450, 1000, 1250, and 1750 mg) may be explored if warranted by emerging safety, PK, pharmacodynamic, biomarker, and response data. Subjects will be evaluated for DLTs during a 21-day DLT-evaluation period.

Part B Dose Expansion may be initiated once the MTD or OBD and a RP2D is established in Part A Dose Escalation and will evaluate the safety, tolerability, PK, pharmacodynamics, and antitumor activity of AZD7789 at the RP2D determined during Part A Dose Escalation in 2 cohorts (Cohorts B1 and B2) described below.

Cohort B1: Subjects with Stage IIIB to IV NSCLC with PD-L1 tumor proportion score (TPS)≥1% who have received one to 2 prior lines of treatment and have anti-PD-1/PD-L1 IO acquired resistance.

Cohort B2: Subjects with Stage IIIB to IV NSCLC with PD-L1 TPS≥50% who have received no prior therapy including IO therapy (ie, IO naïve).

### Example 2: Administration of AZD7789

AZD7789 is administered by IV infusion.

### Example 3: Initial Risk/Benefit Assessment

Studies are conducted to assess risk of AD7789 administration and provide appropriate AZD7789 dose for Parts A and B.

For a 4-week Good Laboratory Practice cynomolgus monkey toxicology study with weekly IV dosing of 100, 200, and 400 mg/kg, the NOAEL was determined to be 100 mg/kg based on the absence of mortalities and adverse clinical findings. Mean plasma AUC data obtained after 4-week repeat dosing at the NOAEL (100 mg/kg/week) provide a 13633-fold safety margin to the predicted AUC at the proposed human starting dose of 2 mg Q3W while the mean Cₘₐₓ gives a 7000-fold safety margin to the predicted Cₘₐₓ at the starting dose of 2 mg Q3W (Table 2).

TABLE 2
Safety Margins of Doses of AZD7789 Used in the Proposed Dose-escalation Scheme

| Dose of | | Predicted | | | Predicted | Safety Margin |
|---|---|---|---|---|---|---|
| AZD7789 | AUC_{cyno} | Human AUCₕ | Safety Margin | C_{max, cyno} | Human C_{max, h} | (C_{max, cyno}/ |
| (mg/75 kg) | (μg · day/mL) | (μg · day/mL) | (AUC_{cyno}/AUCₕ) | (μg/mL) | (μg/mL) | C_{max, h}) |
| 2 | 43626 | 3.2 | 13633 | 3500 | 0.5 | 7000 |
| 7 | | 12.5 | 3490 | | 2.0 | 1750 |
| 22.5 | | 43.0 | 1015 | | 6.6 | 530 |
| 75 | | 149 | 293 | | 22.5 | 156 |
| 225 | | 459 | 95.0 | | 67.9 | 51.5 |
| 750 | | 1555 | 28.1 | | 227 | 15.4 |
| 1500 | | 3136 | 13.9 | | 455 | 7.7 |
| 2000 | | 4193 | 10.4 | | 607 | 5.8 |

Safety margins were calculated based on predicted AUCₕ and C_{max, h} relative to the AUC_{cyno} (43626 μg · day/mL) and C_{max, cyno} (3500 μg/mL) following administration of a 100 mg/kg dose in cynomolgus monkeys.
AUC_{cyno} area under the concentration-time curve from time zero to Day 21 at steady state in cynomolgus monkeys; AUCₕ area under the concentration-time curve from time zero to Day 21 at steady state in humans; C_{max, h} maximal concentration in humans at steady state; C_{max, cyno} maximal concentration in cynomolgus monkeys at steady state.

AZD7789 will be administered at escalating dose intervals with a flat dose 2 mg Q3W. The proposed dose-escalation scheme is 2, 7, 22.5, 75, 225, 750, 1500 to 2000 mg Q3W. Dose escalation for the first 5 dose levels of AZD7789 (ie, 2, 7, 22.5, 75, and 225 mg Q3W) is planned to follow an ATD consisting of 5 single-subject cohorts. Dose escalation for subsequent dose levels of AZD7789 (750, 1500, and 2000 mg) will follow the mTPI-2 algorithm consisting of a minimum of 3 and a maximum of 12 subjects per dose level. If predefined safety criteria are met in an ATD cohort, dose escalation will switch to the mTPI-2 algorithm for all subsequent dose levels.

### Example 4: Study Subjects

This Example provides criteria for selecting subjects for Parts A and B of the study.

Inclusion criteria:
1. Must be 18 years of age or over when enrolled in the study.
2. Histologically or cytologically documented Stage IIIB to IV squamous/non-squamous NSCLC not amenable to curative surgery or radiation as defined below (according to the 8th Edition of the International Association for the Study of Lung Cancer Staging Manual in Thoracic Oncology):
   Part A: Must have received at least one prior line of systemic therapy in the advanced/metastatic setting, of which at least one prior line of therapy contained an approved anti PD-1/PD-L1 therapy and the following:
      (a) The subject had at least 2 administrations of the approved anti-PD-1/PD-L1 therapy alone or in combination with chemotherapy or investigational agents and had IO primary or acquired resistance according to one of the definitions below:
      (i) IO primary resistance (ATD and mTPI-2 cohorts only): Exposure of <6 months to anti PD-1/PD LI therapy.
      (ii) IO acquired resistance (ATD, mTPI-2, and pharmacodynamic backfill cohorts): Exposure of ≥6 months to anti PD-1/PD L1 therapy.
   Part B Cohort B1: Must have received at least one but no more than 2 prior lines of systemic therapy in the advanced/metastatic setting, of which only one prior line of therapy contained an approved anti PD-1/PD-L1 therapy, and the following:
      (a) The most recent treatment included the approved anti-PD-1/PD-L1 therapy with or without platinum based chemotherapy, and the subject had at least 2 administrations of the approved anti-PD-1/PD-L1 therapy and had IO acquired resistance as defined below:
      (i) Exposure of ≥6 months to anti PD-1/PD L1 therapy alone or in combination with chemotherapy with BOR of SD, PR, or CR (such as i/ir/RECIST v1.1) followed by disease progression during treatment or disease progression≤12 weeks after anti PD-1/PD L1 treatment discontinuation.
   Part B Cohort B2: Must not have received any prior systemic therapy in the advanced/metastatic setting. Prior neo/adjuvant therapy not containing any anti PD 1/PD L1 therapy is permitted as long as the subject did not progress for at least 12 months following the last administration.
3. Documented PD-L1 by PD-L1 IHC meeting the criteria below:
   Part A: PD-L1 expression<1% or ≥1% as determined by local report. Note: the number of subjects with PD-L1 expression<1% enrolled in a pharmacodynamic backfill cohort will be ≤50% of the total number of subjects enrolled in that backfill cohort.
   Part B Cohort B1: PD L1 TPS≥1% as determined by local report.
   Part B Cohort B2: PD L1 TPS≥50% as determined by local report.
5. Body mass index≥17.
6. Eastern Cooperative Oncology Group (ECOG) performance status of 0 or 1 at enrollment.
7. Predicted life expectancy of ≥12 weeks.
8. Must have at least one measurable lesion according to RECIST v1.1. (a) For subjects who undergo biopsies at screening and/or on treatment, the biopsied lesion must be distinct from any lesion used in the RECIST v1.1 evaluation.
9. Adequate organ and bone marrow function measured within 28 days prior to first dose. The details of the criteria are shown in Table 3.

TABLE 3
Criteria for Adequate Organ and Marrow Function

| Type | Parameter | Value |
|---|---|---|
| Hematological | Hemoglobin | ≥9.0 g/dL (5.59 mmol/L) |
| | | with no blood transfusions |
| | | (packed red blood cells) |
| | | within 28 days prior to first dose |
| | Absolute | ≥1.5 × 10⁹/L (1,500 per mm³) |
| | neutrophil count | |
| | Platelet count | ≥100 × 10⁹/L (100,000 per mm³) |
| | | with no platelet transfusions |
| | | within 28 days prior to first dose |
| Hepatic | Total bilirubin | ≤1.5 × ULN in the absence |
| | | of Gilbert’s syndrome |
| | | ≤3 × ULN if the participant |
| | | has Gilbert’s syndrome |
| | Alanine | ≤3 × ULN |
| | transaminase and | ≤5 × ULN in the case |
| | aspartate transaminase | of liver metastasis |
| Renal | Calculated creatinine | ≥45 mL/minute |
| | clearance by modified | |
| | Cockcroft Gault | |
| | (Rostoker et al, 2007) | |

ULN = upper limit normal.

### Example 5: Part A Dose Escalation

This Example describes the Part A dose escalation study to determine AZD7789 dosing for Part B.

The primary objective of this study is to investigate the safety and tolerability and thereby identify the MTD or OBD and a RP2D of AZD7789 for further evaluation. Hence the number of subjects has been based on the desire to obtain adequate tolerability, safety, PK, efficacy and biological data while exposing as few subjects as possible to the study intervention and study procedures.

The number of subjects treated during Part A Dose Escalation will depend upon the toxicities observed as the study progresses. With one subject treated at each ATD dose level and a maximum of 12 subjects (including pharmacodynamic backfill) treated at each mTPI-2 dose level, up to 41 subjects may be treated for the 8 planned AZD7789 dose levels (5 dose levels for ATD and 3 dose levels for mTPI-2). Additional subjects may be required if additional dose levels or dosing schedules are explored. Given that the mTPI-2 algorithm leads to a dose-escalation decision if there is no DLT in the first 3 subjects treated in a dose-level cohort, the actual sample size evaluated may be smaller than 41 subjects.

The secondary objectives of the Part A dose escalation include determining the preliminary anti-tumor activity of AZD7789 in subjects with advanced or metastatic tumors. The endpoints include, according to RECIST v1.1: ORR, disease control rate (DCR), duration of response (DoR), and durable response rate (DRR).

For both Part A and Part B, the secondary objectives include assessing the pharmacokinetic (PK) profile compatibility of AZD7789 with Q3W dosing in subjects with advanced or metastatic tumors. The endpoints include serum concentrations and PK parameters (where applicable) of AZD7789; PK parameters to be evaluated include but not limited to maximum observed concentration (Cₘₐₓ), area under the concentration-time curve (AUC), clearance, and terminal elimination half-life (t_{½}).

For both Part A and Part B, the secondary objectives also include assessing the immunogenicity of AZD7789. The endpoints include Incidence of anti-drug antibodies (ADAs) against AZD7789 in serum.

Dose escalation for the first 5 dose levels of AZD7789 (2, 7, 22.5, 75, and 225 mg) is planned to follow an ATD consisting of 5 single-subject cohorts. Dose escalation for subsequent dose levels of AZD7789 (750, 1500, and 2000 mg) will follow the mTPI-2 algorithm consisting of a minimum of 3 and a maximum of 12 subjects per dose level. If predefined safety criteria are met in an ATD cohort, dose escalation will switch to the mTPI-2 algorithm for all subsequent dose levels. Intermediate dose levels may be explored if warranted by emerging safety, PK, pharmacodynamic, biomarker, and response data. Subjects will be evaluated for DLTs during a 21-day DLT-evaluation period.

Based on emerging safety, PK, and pharmacodynamics data, any previously cleared dose level during dose escalation may be expanded up to a total of 12 subjects (the cohort maximum), referred to as pharmacodynamic backfill cohorts. Subjects enrolled into pharmacodynamic backfill cohorts will not impact decisions made by the SRC based on the mTPI-2 algorithm. These pharmacodynamic backfill cohorts will provide additional pharmacodynamics and safety data to inform optimal dose-level selection based on the totality of the data. At the discretion of the Sponsor, additional subjects may be added to previously cleared ATD dose levels to support PK characterization.

Following declaration of the MTD, the dosing cohort enrolling subjects at the MTD may be expanded to the maximum of 12 subjects.

Subjects will receive AZD7789 Q3W administered via IV infusion at the selected dose starting on Cycle 1 Day 1. All subjects will be treated until disease progression, unacceptable toxicity, withdrawal of consent, or other reason for discontinuation occurs.

### Example 6: Part B Dose Expansion

This Example describes the Part B dose expansion study.

Part B Dose Expansion may be initiated once the MTD or OBD and a RP2D is established in Part A Dose Escalation and will evaluate the safety, tolerability, PK, pharmacodynamics, and antitumor activity of AZD7789 at the RP2D determined during Part A Dose Escalation in 2 cohorts (Cohorts B1 and B2) described below.

Cohort B1: Approximately 20 subjects with Stage IIIB to IV NSCLC with PD-L1 TPS≥1% who have received 1 to 2 prior lines of treatment and have anti-PD-1/PD-L1 IO acquired resistance.

Cohort B2: Approximately 20 subjects with Stage IIIB to IV NSCLC with PD-L1 TPS≥50% who have received no prior therapy including IO therapy (ie, IO naïve).

Subjects will receive AZD7789 Q3W administered via IV infusion at the RP2D (determined during Part A Dose Escalation) starting on Cycle 1 Day 1. All subjects will be treated until disease progression, unacceptable toxicity, withdrawal of consent, or other reason for discontinuation occurs.

Cohorts B1 and B2 may opened for enrollment either in parallel or sequentially.

The primary objectives of the Part B dose expansion phase include assessing safety and tolerability of AZD7789 in subjects with advanced or metastatic solid tumors. The endpoints include percentage of subjects with AEs and imAEs, SAEs, DLT-like events, vital signs, and abnormal laboratory parameters; and the rate of AZ7789 discontinuation due to toxicity.

The primary objects of the Part B dose expansion also include determining the preliminary anti-tumor activity of AZD7789 in subjects with advanced or metastatic solid tumors. The endpoints include Objective response rate (ORR) according to Response Evaluation Criteria in Solid Tumors (RECIST) v1.1.

The secondary objectives of the Part B dose expansion include determining the preliminary anti-tumor activity of AZD7789 in subjects with advanced or metastatic solid tumors. The endpoints include According to RECIST v1.1: DCR, DOR, DRR, and progression-free survival (PFS).

### Example 7: Preliminary Part A Results

Background: AZD7789 is a monovalent, bispecific, humanized IgG1 that blocks PD-1 and T cell immunoglobulin and mucin domain 3 (TIM-3), receptors that inhibit antitumor T cell activity and myeloid activation. Dual blockade of PD-1 and TIM-3 can overcome or delay anti-PD-(L)1 resistance. This Example reports dose escalation results from a Phase 1/2a, first-in-human, multicentre, open-label trial of AZD7789 monotherapy in subjects with stage IIIB-IV NSCLC with ≥1 prior line of systemic therapy including ≥1 anti-PD-(L)1 agent.

Methods: Eligible subjects were ≥18 yr old with ECOG PS 0-1. Dose cohorts ranged from 2-225 mg IV Q3W using an accelerated titration design, and from 750-2000 mg using mTPI-2. Primary objective was safety, including dose-limiting toxicities (DLTs). Secondary/exploratory objectives included preliminary efficacy, pharmacokinetics (PK), pharmacodynamics (PD-1 receptor occupancy [RO]), and immunogenicity.

Results: In total, 39 subjects received AZD7789 2-2000 mg; median age 66 yr, 56.4% male, 33.3% PD-L1 status≥1%, 28.2% PD-L1 status≥50%, and 79.5% acquired resistance to prior anti-PD-(L)1 therapy (exposure of ≥6 mo of treatment). Treatment emergent AEs (TEAEs) occurred in 82.1% of subjects and were Grade (G)≥3 in 23.1%; the most common any-grade TEAE was blood creatinine increased (17.9%). No TEAE led to discontinuation. Treatment related AEs (TRAEs) occurred in 41.0% of subjects; the most common was asthenia (7.7%). There were no DLTs or G≥3 TRAEs. Adverse events are summarized in Table 4.

TABLE 4
Summary of Adverse events

| | Number (%) of subjects | | | | | | | | | |
|---|---|---|---|---|---|---|---|---|---|---|
| | ATD | | | | | | | | | |
| | cohorts | | mTPI-2 cohorts | | | | | | | |
| | A1 to A5 | | A6 | | A7 | | A8 | | | |
| | (2-225 | | (750 | | (1500 | | (2000 | | | |
| | mg) | | mg) | | mg) | | mg) | | Total | |
| AE category | N = 8 | | N = 12 | | N = 11 | | N = 3 | | N = 39 | |
| Any AE | 7 | (87.5) | 10 | (83.3) | 8 | (72.7) | 7 | (87.5) | 32 | (82.1) |
| AZD7789- | 3 | (37.5) | 4 | (33.3) | 4 | (36.4) | 5 | (62.5) | 16 | (41.0) |
| related AE | | | | | | | | | | |
| AE of CTCAE | 2 | (25.0) | 3 | (25.0) | 3 | (27.3) | 1 | (12.5) | 9 | (23.1) |
| grade ≥3 | | | | | | | | | | |
| AZD7789- | 0 | | 0 | | 1 | (9.1) | 0 | | 1 | (2.6) |
| related | | | | | | | | | | |
| AE of CTCAE | | | | | | | | | | |
| grade ≥3 | | | | | | | | | | |
| Any SAE | 2 | (25) | 3 | (25.0) | 4 | (36.4) | 1 | (12.5) | 10 | (25.6) |
| AZD7789- | 0 | | 0 | | 2 | (18.2) | 0 | | 2 | (5.1) |
| related SAE | | | | | | | | | | |
| AEs of Special | 5 | (62.5) | 7 | (58.3) | 4 | (36.4) | 3 | (37.5) | 19 | (48.7) |
| Interest | | | | | | | | | | |
| Dis- | 0 | | 0 | | 0 | | 0 | | 0 | |
| continuations | | | | | | | | | | |
| due to AEs | | | | | | | | | | |
| Dose delays | 2 | (25) | 2 | (16.7) | 1 | (9.1) | 0 | | 5 | (12.8) |
| due to AEs | | | | | | | | | | |

| | | | | | |
|---|---|---|---|---|---|
| Death | 0 | 0 | 0 | 0 | 0 |

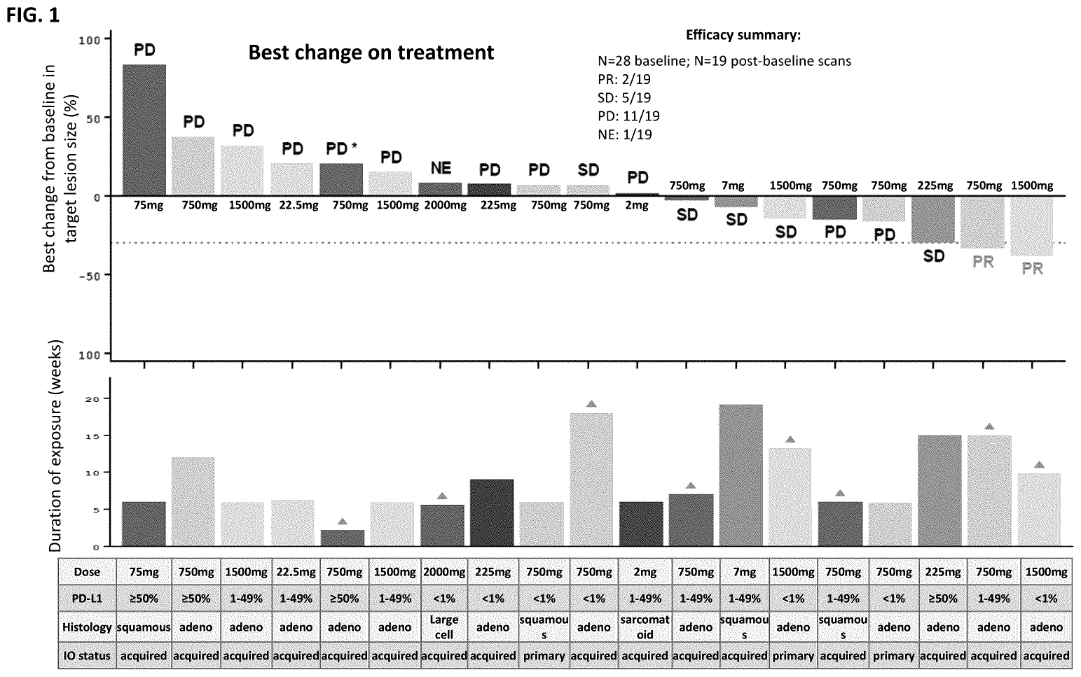

Efficacy was evaluable in 28 subjects, with stable disease≥5 week in 7 (25.0%), including 2 unconfirmed partial responses, and progression in 11 (39.3%). Tumor shrinkage was seen in 8 subjects. As of this report, 24 subjects remained on AZD7789. PK was generally dose proportional, with t_{½}˜7 days; antidrug antibodies had limited impact on PK. Doses≥225 mg led to PD-1 RO>90% on peripheral T cells. A waterfall plot of post-baseline scans of 18 of the subjects is shown in FIG. 1.

PK results are in line with predictions and are dose proportional for a total of 21 subjects tested. The observed half-life of AZD7789 is approximately 7 days. Anti-drug antibodies were observed in 11 of 21 subjects, with one subject at a 750 mg having anti-drug antibodies showing a visible impact on PK.

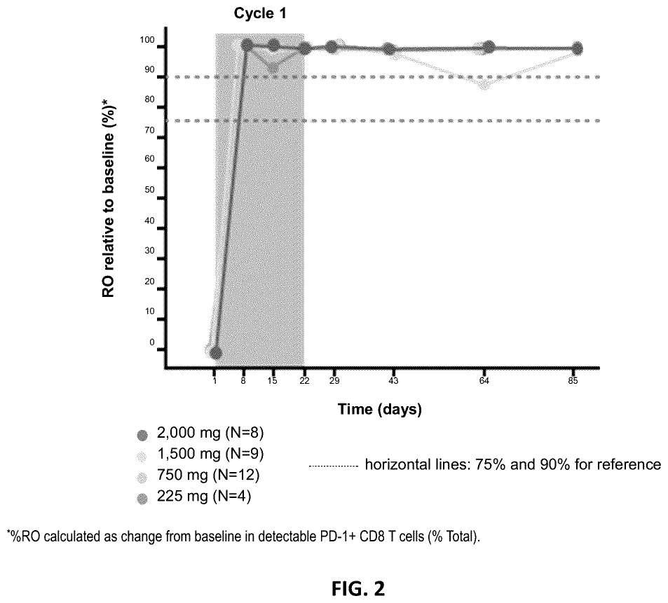

FACs data show that doses of greater than 225 mg achieve durable PD-1 receptor occupancy of greater than 90% in IO pre-treated NSCLC patients. Percent receptor occupancy relative to baseline is plotted in FIG. 2 for doses greater than 225 mg as shown. Signals of peripheral T cells and myeloid activation are observed at 750 mg and 1500 mg but the current data does not discern between the two doses.

Conclusions: AZD7789 has manageable safety and shows preliminary efficacy at tolerable doses. Evaluation is ongoing in immunotherapy-naive subjects with NSCLC and subjects with acquired resistance to immunotherapy.

### Example 8: Hodgkin Lymphoma Study

The cellular microenvironment of classical Hodgkin Lymphoma (cHL) is characterized by a paucity of B cell-derived malignant Hodgkin Reed-Sternberg (HRS) cells within a tumor microenvironment (TME) comprised of non-malignant stromal and immune cells. Despite extensive immune cell infiltration, the antitumor immune response is ineffective. It is hypothesized that this is due to the ability of HRS cells to modulate the TME to avoid immune cell recognition and killing.

Hodgkin Reed-Sternberg cells co-opt the programmed cell death protein-1 (PD-1) pathway and upregulate PD-1 ligands by several mechanisms including copy gain alterations of 9p24.1. Topographical analysis of cHL TME demonstrated that HRS cells with programmed cell death-ligand 1 (PD-L1) overexpression were in close proximity to PD-1⁺CD4⁺T cells within the TME. Furthermore, reports demonstrated that PD-1 expression on CD4⁺T cells, within the cHL TME, is more frequently observed on the T helper 1 (Th1) effector cells compared to CD4⁺Tregs, which are in turn largely PD-1 negative. Consequently, a double immune evasion strategy exists within cHL, whereby Th1-mediated immune responses are likely dysfunctional while the immunosuppressive Treg cells retain functionality.

Due to the strong scientific rationale, anti-PD-1/PD-L1 therapy entered clinical trials and yielded a breakthrough in relapsed or refractory (r/r) cHL. However, as monotherapy, less than 20% of patients achieved a complete response (CR) and results were not durable.

Although the targeting of the PD-1 axis shows clinical potential, the relative lack of complete responders suggests additional mechanisms contribute to promote HRS survival. T cell immunoglobulin and mucin domain-containing protein-3 (TIM-3), is an immune checkpoint (IC) that has been associated with dysfunctional T cells and has recently been implicated as a resistance mechanism following PD-1 therapy. While data in cHL is limited, TIM-3 expression has been observed in cHL. Therefore, co-targeting both PD-1 and TIM-3 in cHL may reinvigorate immune responses and lead to more durable antitumor activity.

This study will investigate the safety, tolerability, pharmacokinetics (PK), and antitumor activity of AZD7789, a PD-1/TIM-3 bispecific monoclonal antibody (mAb), in the r/r cHL population. The study design comprises of two parts: Part A consists of a dose escalation that will enroll r/r cHL subjects and Part B a dose expansion, which will enroll in Cohort B1 r/r cHL subjects who were previously exposed to anti-PD-1/PD-L1 based therapy and in Cohort B2 r/r cHL subjects who are naïve to anti-PD-1/PD-L1 therapy.

Primary and secondary objectives for the study are similar to those for the NSCLC study discussed in the above Examples.

Disease response will be assessed according to Investigator assessment using Modified Lugano (2014) and RECIL criteria. Disease response will be assessed according to Blinded Independent Central Review using Modified Lugano (2014).

### Study Design

This Phase I/II, open-label multi-center, dose escalation and dose expansion study will evaluate the safety, tolerability, PK, pharmacodynamics, and antitumor of AZD7789 in adult/young adult subjects with r/r cHL. The study includes two parts: Part A Dose Escalation and Part B Dose Expansion. Part A will enroll subjects with r/r cHL previously treated with anti-PD-1/PD-L1 based therapy. Part B will be subdivided into Cohort B1, enrolling a patient population identical to Part A, and Cohort B2 enrolling subjects who never received an anti-PD-1/PD-L1 therapy previously.

Part A Dose Escalation will evaluate up to 8 dose levels of AZD7789 and consist of up to 52 subjects with r/r cHL. Cohorts A1 to A4 will be single subject cohorts with 4 dose levels of AZD7789 (2, 7, 22.5, and 75 mg) based on an accelerated titration design (ATD).

However, the operating model will switch to a modified toxicity probability interval-2 (mTPI-2) algorithm should a DLT or Grade 2 or greater treatment-emergent adverse event (TEAE) occur.

Cohorts A5, A6, and A7 for subsequent dose levels of AZD7789 (225, 750, and 1500 mg) will enroll 3 subjects based on the mTPI-2 algorithm to establish the safety of AZD7789. Based on emerging data, the optional Cohort A8 (at 2000 mg) may open and may also enroll up to 12 subjects. After a cohort is declared safe (eg, Cohort A5, A6, A7 or A8) and based on emerging safety, efficacy, PK and biomarker data, the cohort may be expanded (up to 12 subjects); the cohorts may be opened in parallel to fill their rosters of up to 12 subjects each (mini dose expansion). Intermediate dose levels (50, 150, 450, 1000, 1250, and 1750 mg) may be explored if warranted by emerging safety, PK, pharmacodynamics, biomarker, and response data.

The planned starting dose will be 2 mg (dose level 1). Subjects will be evaluated for DLTs during a 28-day DLT evaluation period.

Part B Dose Expansion may be considered once a recommended Phase 2 dose (RP2D) is determined in Part A Dose Escalation and will evaluate the safety, tolerability, PK, pharmacodynamic, and antitumor activity of AZD7789 at the determined RP2D in 2 cohorts (Cohorts B1 and B2) described below.

Cohort B1: Approximately 88 subjects with cHL who have received at least 2 prior lines of systemic treatment and have subsequently relapsed or become refractory to treatment and have been previously treated with at least 3 cycles of anti-PD-1/PD-L1 based therapy. Subjects in Cohort B1 will be identical to those of the dose escalation stage. For this reason, enrolment of a larger sample size will provide a clearer confirmatory efficacy signal. For the analysis in Cohort B1, subjects treated at the RP2D in Part A (N=12) will be combined with subjects enrolled in Part B, which gives a total sample size of approximately up to 100.

Cohort B2: Approximately 40 subjects with cHL who have received at least 2 prior lines of systemic treatment and have subsequently relapsed or become refractory to treatment, but have not received an anti-PD-1/PD-L1 based therapy.

### Example 9: Expanded Results of Part A Dose Escalation Study

The Part A dose escalation study of AZD7789 described above was continued. Inclusion criteria for subjects in Part A was as provided in Example 4 above. Abbreviations are as defined in Example 1 above. Forty five subjects were placed in 8 cohorts (including backfill) as summarized in Table 5.

TABLE 5
Part A Cohorts (n = 45)

| | AZD7789 | |
|---|---|---|
| Cohort | Dose (n) | Backfill |
| A1 | 2 mg (n = 1) | |
| A2 | 7 mg (n = 1) | |
| A3 | 22.5 mg (n = 1) | |
| A4 | 75 mg (n = 1) | |
| A5 | 225 mg (n = 1) | 225 mg (n = 3) |
| A6 | 750 mg (n = 5) | 750 mg (n = 7) |
| A7 | 1500 mg (n = 4) | 1500 mg (n = 9) |
| A8 | 2000 mg (n = 8) | 2000 mg (n = 4) |

AZD7789 was administered intravenously once every three weeks (Q3W). The demographics of the subjects are provided in Table 6.

TABLE 6
Baseline Demographics and Disease Characteristics

| | AZD7789 2-2,000 |
|---|---|
| | mg IV Q3W |
| | (N = 45) |
| Median age (range), years | 66.0 (44-79) |
| Female/Male, n (%) | 21 (46.7)/24 (53.3) |
| Asian/Black or African American/ | 1 (2.2)/1 (2.2)/27 (60.0)/ |
| White/Other/Missing, n (%) | 2 (4.4)/14 (31.1) |
| ECOG PS 0/1, n (%) | 14 (31.1)/31 (68.9) |
| Disease stage III/IV/Missing, n (%) | 1 (2.2)/43 (95.6)/1 (2.2) |
| Histology, n (%): | |
| Adenocarcinoma/Squamous/ | 34 (75.6)/9 (20.0)/ |
| Large cell carcinoma/Other | 1 (2.2)/1 (2.2) |
| PD-L1 expression, n (%): | |
| <1%/1-49%/≥50%/Missing | 18 (40.0)/14 (31.1)/ |
| | 12 (26.7)/1 (2.2) |
| Resistance to prior | |
| anti-PD-(L)1 treatment, n (%): | |
| Primary/Acquired/Missing* | 6 (13.3)/ |
| | 35 (77.8)/4 (8.9) |
| Prior chemotherapy, n (%) | 42 (93.3) |
| Prior lines of treatment, median (range) | 2.0 (1-6) |

As can be seen in Table 6, most subjects had acquired resistance to therapies targeting PD-1 or PD-L1.

The median duration of treatment was 12 weeks as of the time of data collection. There were no DLTs observed and no subjects discontinued AZD7789 due to AEs. Treatment-emergent adverse events (TEAEs) occurred in 95.6% of subjects and were grade 3 or higher in 40.0%. Treatment-related adverse events (TRAEs) occurred in 60.0% of subjects. A safety summary of the study is provided in Table 7.

TABLE 7
Safety Summary

| | Cohort | Cohort | Cohort | Cohort | Cohort | Cohort | Cohort | Cohort | Total |
|---|---|---|---|---|---|---|---|---|---|
| | A1 | A2 | A3 | A4 | A5 | A6 | A7 | A8 | 2- |
| | 2 mg | 7 mg | 22.5 mg | 75 mg | 225 mg | 750 mg | 1,500 mg | 2,000 mg | 2,000 mg |
| n (%) | (n = 1) | (n = 1) | (n=1) | (n = 1) | (n = 4) | (n = 12) | (n = 13) | (n = 12) | (N = 45) |
| Any AE | 0 | 1 | 1 | 1 | 4 | 12 | 12 | 12 | 43 |
| | | (100.0) | (100.0) | (100.0) | (100.0) | (100.0) | (92.3) | (100.0) | (95.6) |
| Any AE possibly | 0 | 1 | 1 | 0 | 2 | 6 | 9 | 8 | 27 |
| related to AZD7789 | | (100.0) | (100.0) | | (50.0) | (50.0) | (69.2) | (66.7) | (60.0) |
| Any Grade ≥3 AE | 0 | 1 | 0 | 0 | 1 | 5 | 5 | 6 | 18 |
| | | (100.0) | | | (25.0) | (41.7) | (38.5) | (50.0) | (40.0) |
| Any Grade ≥3 AE | 0 | 0 | 0 | 0 | 0 | 0 | 1 | 0 | 1 |
| possibly related to | | | | | | | (7.7) | | (2.2) |
| AZD7789 | | | | | | | | | |
| Any treatment- | 0 | 1 | 0 | 0 | 1 | 5 | 5 | 3 | 15 |
| emergent SAE | | (100.0) | | | (25.0) | (41.7) | (38.5) | (25.0) | (33.3) |
| Any treatment- | 0 | 0 | 0 | 0 | 0 | 0 | 1 | 0 | 1 |
| emergent SAE | | | | | | | (7.7) | | (2.2) |
| possibly related to | | | | | | | | | |
| AZD7789 | | | | | | | | | |
| Any AE leading to | 0 | 0 | 0 | 0 | 10 | 0 | 0 | 0 | 0 |
| death | | | | | | | | | |
| Any AE leading to | 0 | 0 | 0 | 0 | 0 | 0 | 0 | 0 | 0 |
| discontinuation | | | | | | | | | |
| Any immune- | 0 | 1 | 0 | 0 | 2 | 2 | 5 | 6 | 16 |
| mediated AE | | (100) | | | (50) | (16.7) | (38.5) | (50.0) | (35.6) |
| (imAE) | | | | | | | | | |

The most common TEAEs of all grades were decreased appetite, nausea, increased blood creatinine increased (24.4% each), and fatigue (22.2%). The most common Grade≥3 TEAEs were decreased haemoglobin and uncoded events (6.7% each), and anaemia, dyspnoea, and asthenia (4.4% each). Two subjects had treatment-emergent Grade≥3 dyspnoea, one of which is considered treatment-related. This was the only Grade≥3 TRAE reported. There were no Grade 4 or 5 TRAEs. The TEAEs are summarized in Table 8.

TABLE 8
Treatment-emergent AEs occurring in ≥15% of
subjects at any Grade, or in ≥4% of subjects at Grade ≥3

| n (%) | Any Grade | Grade ≥3 |
|---|---|---|
| Any AE | 43 (95.6) | 18 (40.0) |
| Decreased appetite | 11 (24.4) | 1 (2.2) |
| Nausea | 11 (24.4) | 0 |
| Blood creatinine increased | 11 (24.4) | 0 |
| Fatigue | 10 (22.2) | 1 (2.2) |
| Cough | 8 (17.8) | 0 |
| Anaemia | 7 (15.6) | 2 (4.4) |
| Dyspnoea | 6 (13.3) | 2 (4.4) |
| Asthenia | 6 (13.3) | 2 (4.4) |
| Haemoglobin decreased | 4 (8.9) | 3 (6.7) |
| Uncoded events | 3 (6.7) | 3 (6.7) |

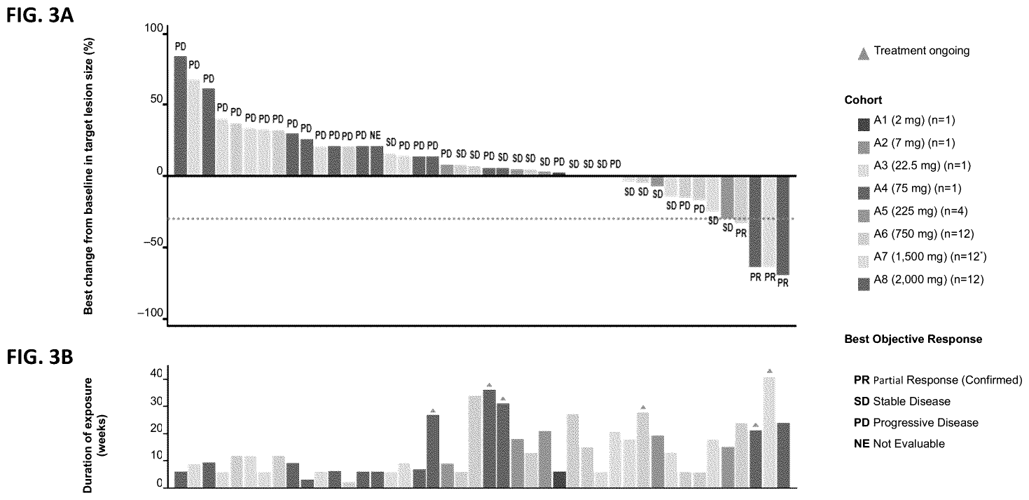

Lesion size was measured in 44 of the subjects. One subject in cohort A7 had not yet been scanned. A waterfall plot of percent change from baseline in target lesion size is shown in FIG. 3A. Objective responses are also provided in the plot, indicating a complete response (CR), a partial response (PR), stable disease (SD), progressive diseased (PD) or not evaluable (NE). The duration of exposure in weeks for each subject is plotted in FIG. 3B. There were four confirmed partial responses, all at doses of 750 mg or higher. Stable disease for at least 5 weeks was observed in 16 subjects across several dose levels.

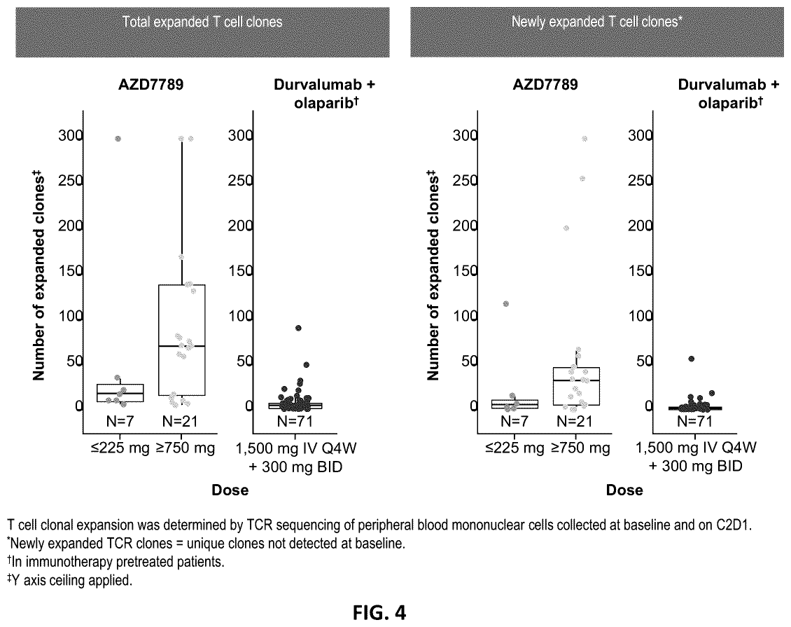

T cell receptor sequencing was performed to determine clonal expansion in subjects before and after treatment with AZD7789 or a combination of durvalumab (a PD-L1 inhibitor)+olaparib from a separate study (Iyer, et al. Cancer Res. 2023;83:CT039). The box plots in FIG. 4 show the number of expanded clones for each treatment. Both total and new T cell receptor clonal expansion were greater in subjects treated with AZD7789 than in immunotherapy pretreated subjects who received durvalumab. Expansion of both existing and new T cell clones supports a myeloid-T cell-driven mechanism of action.

In conclusion, this study demonstrated that:
AZD7789 was tolerable at all doses tested with no observed DLTs; doses≥225 mg resulted in sustained >90% occupancy of PD-1 receptors on peripheral T cells.
In dose escalation, clinical activity was observed in IO pretreated (e.g., IO-resistant) subjects with NSCLC.
There were 4 subjects with confirmed partial responses at doses above ≥750 mg.

AZD7789 drives expansion of both existing and new peripheral T cells.

### Example 10: Additional Results from Part A Dose Escalation Study

Additional studies for the Part A dose escalation study of AZD7789 described above were performed. Inclusion criteria for subjects in Part A was as provided in Example 4 above. Abbreviations are as defined in Example 1 above. Thirty two subjects were placed in 8 cohorts as summarized in Table 9.

TABLE 9
Part A Cohorts (n = 32)

| | AZD7789 |
|---|---|
| Cohort | Dose (n) |
| A1 | 2 mg (n = 1) |
| A2 | 7 mg (n = 1) |
| A3 | 22.5 mg (n = 1) |
| A4 | 75 mg (n = 1) |
| A5 | 225 mg (n = 1) |
| A6 | 750 mg (n = 12) |
| A7 | 1500 mg (n = 11) |
| A8 | 2000 mg (n = 0) |

AZD7789 was administered intravenously once every three weeks (Q3W). Patients were predominantly male (71.9%) with Stage IV disease (65.6%) and had a median age of 38.0 years. They had received a median of 5.5 prior lines of therapy. The demographics of the subjects are provided in Table 10.

TABLE 10
Baseline Demographics and Disease Characteristics

| | N = 32 | |
|---|---|---|
| Median age (range), years | 38.0 | (21-77) |
| Male/female, n (%) | 23 (71.9)/9 (28.1) | |
| Race, n (%) | | |
| White | 22 | (68.8) |
| Asian | 2 | (6.3) |
| Black or African American | 2 | (6.3) |
| Native Hawaiian or | 1 | (3.1) |
| Other Pacific Islander | | |
| Other | 1 | (3.1) |
| Missing or not reported | 4 | (12.5) |
| Disease stage, n (%) | | |
| I | 1 | (3.1) |
| II | 5 | (15.6) |
| III | 5 | (15.6) |
| IV | 21 | (65.6) |
| Disease subtype at | | |
| study entry, n (%) | | |
| Nodular sclerosing | 20 | (62.5) |
| Mixed cellularity | 4 | (12.5) |
| Lymphocyte rich | 2 | (6.3) |
| Lymphocyte depleted | 1 | (3.1) |
| Unknown | 5 | (15.6) |
| Bulky disease, n (%)* | 4 | (12.5) |
| Extranodal disease, n (%) | 18 | (56.3) |
| Prior anticancer therapy | 5.5 | (3-13) |
| lines, median (range) | | |
| Prior disease-related | | |
| treatment modalities, n (%) | | |
| Anti-PD-1 | 32 | (100) |
| Anti-PD-L1 | 1 | (3.1) |
| ASCT | 19 | (59.4) |
| Brentuximab | 29 | (90.6) |

*Defined as a single nodal mass with longest diameter of 10 cm or > 1/3 the transthoracic diameter at any level of thoracic vertebrae as measured by computed tomography scan

The median duration of exposure to AZD7789 was 9.2 weeks (range, 0.6-51.3) with a median of 3 cycles received (range 1-17). AZD7789 was well tolerated (Table 11). The most common treatment-emergent AEs were fatigue and headache (21.9% each) (Table 12). One Grade≥3 treatment-emergent AE was fatal: sepsis secondary to gastric ulcer rupture, not related to AZD7789 but deemed a dose-limiting toxicity (DLT) by the safety review committee. No other DLTs were reported. One patient had a treatment-related Grade≥3 AE: decreased neutrophil count. No treatment-related AEs led to death or discontinuation of AZD7789

TABLE 11
Safety Summary

| | N = 32 | | | |
|---|---|---|---|---|
| | Treatment- | | Possibly related | |
| Safety, n (%) | emergent | | to AZD7789 | |
| Any AE | 26 | (81.3) | 20 | (62.5) |
| Grade ≥3 AE | 3 | (9.4) | 1 | (3.1)** |
| AE with outcome of death | 1 | (3.1)* | 0 | |
| AE leading to discontinuation of | 1 | (3.1) | 0 | |
| AZD7789 | | | | |
| Serious AE | 7 | (21.9) | 4 | (12.5)^{†} |
| AESI | 10 | (31.3) | 8 | (25.0)^{‡} |
| Grade ≥3 AESI | 0 | | 0 | |
| Immune-mediated AE | 3 | (9.4)^{§} | 3 | (9.4)^{§} |

Patients with multiple events are counted only once per applicable category
*Grade 5 sepsis (n = 1);
**Grade 3 neutrophil count decreased (n = 1);
^{†}Grade 1 cytokine release syndrome (n = 1); Grade 1 infusion-related reaction (n = 1); Grade 1 not coded (n = 1,); and Grade 2 herpes zoster infection (n = 1);
^{‡}Grade 1 facial rash (n = 1); Grade 1 cytokine release syndrome (n = 1); Grade 1 infusion-related reaction (n = 3); Grade 1 stomatitis mouth sores and Grade 1 pruritic rash (n = 1); Grade 2 eczema (n = 1); Grade 1 erythema (n = 1); Grade 2 autoimmune thyroiditis (n = 1); and Grade 1 dermatitis acneiform (n = 1);
^{§}Grade 1 pruritic rash (n = 1); Grade 1 not coded (n = 1); Grade 2 lipase increased and grade 2 autoimmune thyroiditis (n = 1)
AE, adverse event;
AESI, AE of special interest

TABLE 12
Treatment-emergent AEs occurring at any Grade
in ≥5% of patients or at Grade ≥3 in any patients

| Treatment-emergent AEs, n (%) | Any Grade | Grade ≥3 |
|---|---|---|
| Any AE | 26 (81.3) | 3 (9.4)* |
| Fatigue | 7 (21.9) | 0 |
| Headache | 7 (21.9) | 0 |
| Back pain | 4 (12.5) | 0 |
| Infusion-related reaction | 4 (12.5) | 0 |
| Nausea | 4 (12.5) | 0 |
| Alanine aminotransferase increased | 3 (9.4) | 0 |
| Anemia | 3 (9.4) | 0 |
| Chills | 3 (9.4) | 0 |
| Dizziness | 3 (9.4) | 0 |
| Pyrexia | 3 (9.4) | 0 |
| Abdominal pain | 2 (6.3) | 1 (3.1) |
| Asthenia | 2 (6.3) | 0 |
| TSH increased | 2 (6.3) | 0 |
| Diarrhea | 2 (6.3) | 0 |
| Nasal congestion | 2 (6.3) | 0 |
| Sinus bradycardia | 2 (6.3) | 0 |
| Gastric perforation | 1 (3.1) | 1 (3.1) |
| Neuralgia | 1 (3.1) | 1 (3.1) |
| Neutrophil count decreased | 1 (3.1) | 1 (3.1) |
| Sepsis | 1 (3.1) | 1 (3.1) |
| Not coded | 2 (6.3) | 0 |

*One patient had Grade 3 abdominal pain, Grade 4 gastric perforation, and Grade 5 sepsis (none considered related to AZD7789); one patient had Grade 3 neuralgia (not considered related to AZD7789); and one patient had Grade 3 neutrophil count decreased (considered related to AZD7789)
AE, adverse event;
TSH, thyroid stimulating hormone.

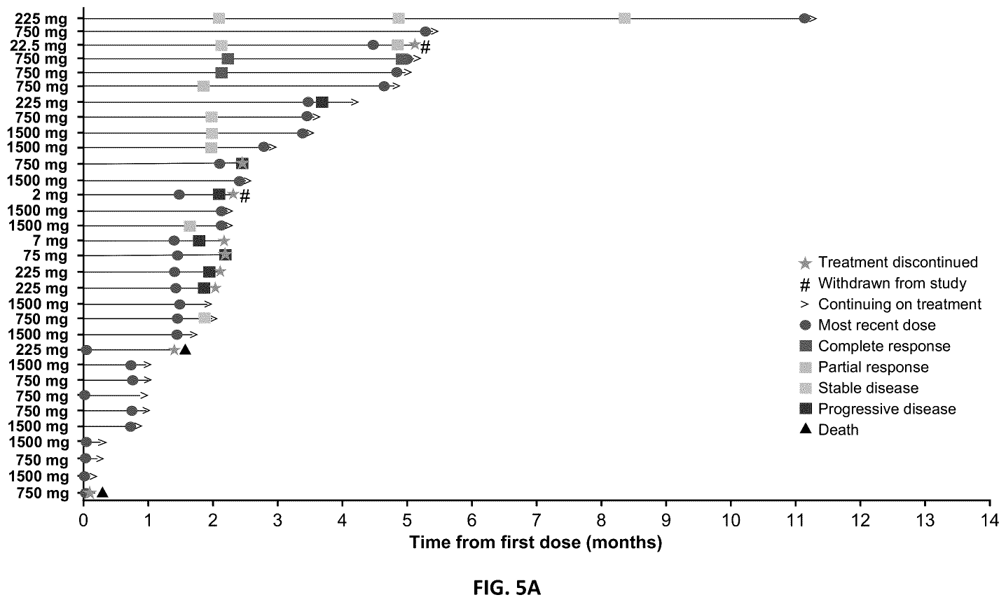

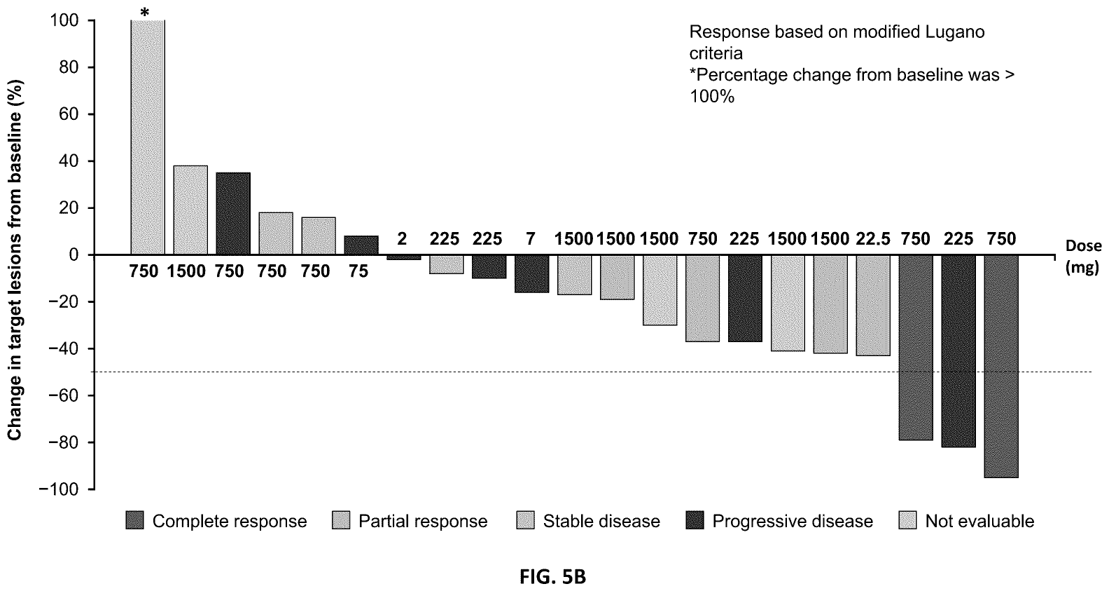

Lesion size was measured in 21 of the subjects. The response and duration of response are plotted in FIG. 5A. Of the 21 patients who had their first disease assessment, 6 had an objective response based on modified Lugano criteria. Objective responses included:
1/1 patient with a partial response (PR) in Cohort A3 (22.5 mg)
3/7 patients in Cohort A6 (750 mg): 2 with complete responses (CR) and 1 with a PR
2/6 patients with PRs in Cohort A7 (1500 mg)

Objective responses are shown in the waterfall plot of FIG. 5B. Of these 21 patients, 4 had stable disease at doses ranging from 7-1500 mg and 4 were not evaluable.

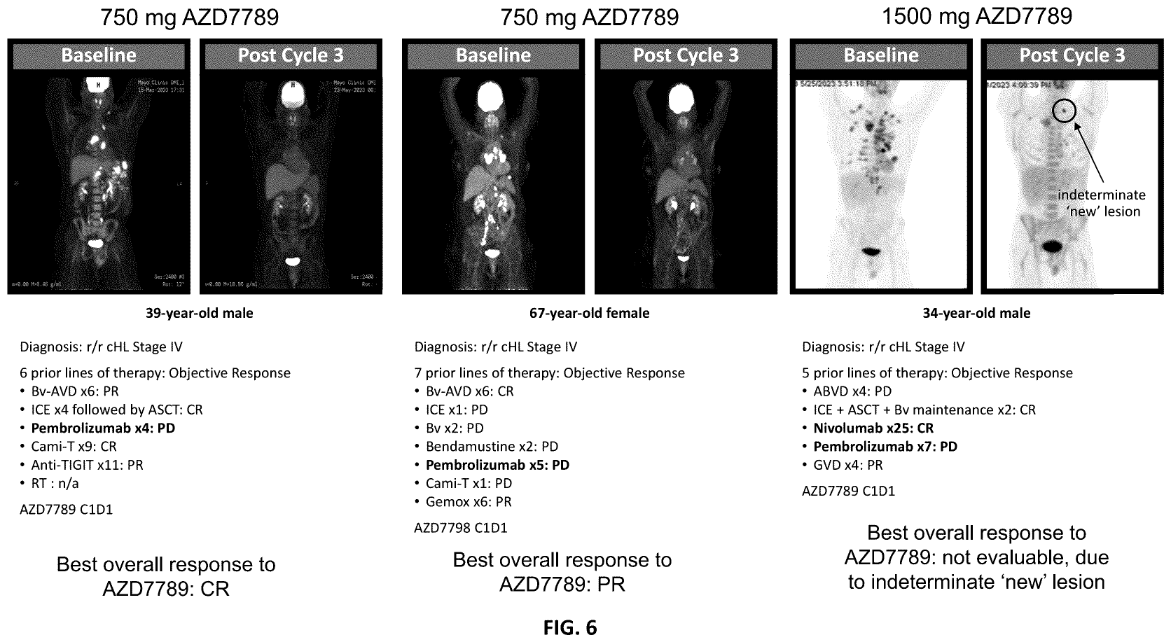

Responders included patients who were refractory or relapsed on prior anti-PD-1 treatment as summarized in FIG. 6. As shown in FIG. 6, three responders were given full body scans at baseline prior to therapy and after completion of cycle 3 of treatment. Responder 1 was a 39 year old male with 6 prior lines of therapy as shown; Responder 2 was a 67 year old female with 7 prior lines of therapy as shown; and Responder 3 was a 34 year old male with 5 prior lines of therapy as shown.

Consistent with prior results, AZD7789 doses≥225 mg resulted in sustained PD-1 receptor occupancy (≥90%) on peripheral T cells.

AZD7789 pharmacokinetics (PK) were studied in 22 patients at doses up to 1500 mg. PK was broadly dose-proportional. The half-life of AZD7789 was approximately 9 days. A summary of the PK results is shown in Table 13.

TABLE 12
AZD7789 Pharmacokinetics

| | 2 | 7 | 22.5 | 75 | 225 | 750 | 1500 |
|---|---|---|---|---|---|---|---|
| Cycle 1 | mg* | mg* | mg* | mg* | mg | mg | mg |
| Cₘₐₓ (μg/mL), | NC | 1.410 | 5.800 | 15.41 | 53.06 | 223.0 | 715.2 |
| GM (gCV %) | [n = 0] | (NC) | (NC) | (NC) | (28.22) | (19.53) | (63.98) |
| | | [n = 1] | [n = 1] | [n = 1] | [n = 5] | [n = 7] | [n = 7] |
| AUCₗₐₛₜ | NC | 4.327 | 26.99 | 86.73 | 264.7 | 2039 | 3989 |
| (Day*μg/mL), | [n = 0] | (NC) | (NC) | (NC) | (67.34) | (15.08) | (46.33) |
| GM (gCV %) | | [n = 1] | [n = 1] | [n = 1] | [n = 5] | [n = 6] | [n = 7] |
| t_{1/2} (Day), | NC | 2.965 | 5.033 | 4.486 | 8.757 | 10.81 | 10.6 |
| GM (gCV %) | [n = 0] | (NC) | (NC) | (NC) | (116.3) | (58.72) | (17.07) |
| | | [n = 1] | [n = 1] | [n = 1] | [n = 4] | [n = 6] | [n = 5] |

LLOQ = 0.1 μg/mL
*Median reported due to n < 3
AUC, area under the curve;
GM, geometric mean;
gCV %, geometric coefficient of variation;
LLOQ, lower limit of quantitation;
NC, not calculated

AZD7789 showed low immunogenicity. Treatment-emergent antidrug antibodies (ADAs) were observed in 8/23 (35%) patients studied, who received AZD7789 doses up to 1500 mg. The impact of ADAs titer on PK and receptor occupancy was low.

In conclusion, this study demonstrated that:
AZD7789 was well tolerated, with a manageable safety profile at all doses tested.
Early efficacy data at 750 mg and 1500 mg were encouraging, with objective responses in 5/13 patients who had their first disease assessment, the majority of whom were anti-PD-1 refractory or who had relapsed on prior treatment.
PD-1 receptor occupancy on peripheral T cells was durable at doses≥225 mg, supporting the proposed mechanism of action.
AZD7789 PK was broadly dose-proportional.
Treatment-emergent ADAs had minimal impact on PK and PD-1 receptor occupancy.

1. A method for treating non-small cell lunger cancer (NSCLC) or classical Hodgkin Lymphoma (cHL) in a subject, comprising administering to the subject a bispecific binding protein that specifically binds to Programed Death-1 (PD-1) and T cell immunoglobulin and mucin domain-containing protein 3 (TIM-3) in an amount from about 70 mg to about 1500 mg, the bispecific binding protein comprising:
a) a first binding domain that specifically binds to PD-1, wherein the first binding domain comprises a heavy chain variable domain comprising a HCDR1 having the amino acid sequence of SEQ ID NO: 4, a HCDR2 having the amino acid sequence of SEQ ID NO: 5, and a HCDR3 having the amino acid sequence of SEQ ID NO: 6, and a light chain variable domain comprising a LCDR1 having the amino acid sequence of SEQ ID NO: 10, a LCDR2 having the amino acid sequence of SEQ ID NO: 11 and a LCDR3 having the amino acid sequence of SEQ ID NO: 12; and
b) a second binding domain that specifically binds to TIM-3, wherein the second binding domain comprises a heavy chain variable domain comprising a HCDR1 having the amino acid sequence of SEQ ID NO: 1, a HCDR2 having the amino acid sequence of SEQ ID NO: 2, and a HCDR3 having the amino acid sequence of SEQ ID NO: 3, and a light chain variable domain comprising a LCDR1 having the amino acid sequence of SEQ ID NO: 7, a LCDR2 having the amino acid sequence of SEQ ID NO: 8, and a LCDR3 having the amino acid sequence of SEQ ID NO: 9.

2. The method of claim 1, wherein the amount of the bispecific binding protein administered is about 70 mg, about 150 mg, about 210 mg, about 450 mg, about 750 mg, about 800 mg, about 850 mg, about 900 mg, about 950 mg, about 1000 mg, about 1250 mg, or about 1500 mg.

3. The method of claim 2, wherein the amount of the bispecific binding protein administered is about 750 mg.

4. The method of claim 2, wherein the amount of the bispecific binding protein administered is about 1500 mg.

5. The method of claim 2, wherein the bispecific binding protein is administered once per treatment cycle.

6. The method of claim 5, wherein the treatment cycle is about 7 days, about 14 days, about 21 days, about 28 days, or about 35 days.

7. The method of claim 5, wherein the treatment cycle is about 21 days.

8. The method of claim 7, wherein the treatment cycle is repeated for up to 35 cycles.

9.-12. (canceled)

13. The method of claim 7, wherein the subject has not received a prior line of systemic therapy.

14. The method of claim 1, wherein the subject has not received a prior line of immuno-oncology (IO) therapy.

15. The method of claim 12, wherein the subject has previously received a chemotherapy.

16. The method of claim 1, wherein the subject has previously received an IO therapy.

17.-18. (canceled)

19. The method of claim 1, wherein the subject has IO acquired resistance.

20. (canceled)

21. The method of claim 19, wherein the subject has a radiologically documented tumor progression or clinical deterioration following initial treatment with an anti-PD-1/PD-L1 therapy for a minimum of 3-6 months, as monotherapy or in combination with chemotherapy, and had signs of initial clinical benefit.

22. The method of claim 1, wherein the NSCLC or cHL comprises a NSCLC or cHL cell which expresses PD-L1.

23. The method of claim 1, wherein the first binding domain of the bispecific binding protein that specifically binds to PD-1 comprises a heavy chain variable domain having the amino acid sequence of SEQ ID NO: 19 and a light chain variable domain having the amino acid sequence of SEQ ID NO:21, and wherein the second binding domain of the bispecific binding protein that specifically binds to TIM-3 comprises a heavy chain variable domain having the amino acid sequence of SEQ ID NO:14 and a light chain variable domain having the amino acid sequence of SEQ ID NO:17.

24.-43. (canceled)

44. The method of claim 1, wherein the cancer is non-small cell lung cancer (NSCLC), or wherein the NSCLC is advanced or metastatic, or wherein the NSCLC is squamous or non-squamous NSCLC.

45.-46. (canceled)

47. The method of claim 44, wherein the subject has a PD-L1 tumor proportion score of greater than or equal to 1%, or wherein the subject has a PD-L1 tumor proportion score of greater than or equal to 50%.

48. (canceled)

49. The method of claim 47, wherein the subject is checkpoint inhibitor (CPI) naïve.

50.-68. (canceled)


SEQUENCE LISTING (30 sequences)

SEQ ID NO: 1 - protein, 5 aa, synthetic construct
  REGION 1..5: note = Synthetic: AZD 7789 / TIM-3 / VH CDR1
SYAMS

SEQ ID NO: 2 - protein, 17 aa, synthetic construct
  REGION 1..17: note = Synthetic: AZD 7789 / TIM-3 / VH CDR2
AISGSGGSTYYADSVKG

SEQ ID NO: 3 - protein, 13 aa, synthetic construct
  REGION 1..13: note = Synthetic: AZD 7789 / TIM-3 / VH CDR3
GSYGTYYGNYFEY

SEQ ID NO: 4 - protein, 5 aa, synthetic construct
  REGION 1..5: note = Synthetic: AZD 7789 / PD-1 / VH CDR1
DYGMH

SEQ ID NO: 5 - protein, 17 aa, synthetic construct
  REGION 1..17: note = Synthetic: AZD 7789 / PD-1 / VH CDR2
YISSGSYTIYSADSVKG

SEQ ID NO: 6 - protein, 13 aa, synthetic construct
  REGION 1..13: note = Synthetic: AZD 7789 / PD-1 / VH CDR3
RAPNSFYEYYFDY

SEQ ID NO: 7 - protein, 11 aa, synthetic construct
  REGION 1..11: note = Synthetic: AZD 7789 / TIM-3 / VL CDR1
GGDNIGGKSVH

SEQ ID NO: 8 - protein, 7 aa, synthetic construct
  REGION 1..7: note = Synthetic: AZD 7789 / TIM-3 / VL CDR2
YDSDRPS

SEQ ID NO: 9 - protein, 11 aa, synthetic construct
  REGION 1..11: note = Synthetic: AZD 7789 / TIM-3 / VL CDR3
QVLDRRSDHFL

SEQ ID NO: 10 - protein, 18 aa, synthetic construct
  REGION 1..18: note = Synthetic: AZD 7789 / PD-1 / VL CDR1
SASSKHTNLYWSRHMYWY

SEQ ID NO: 11 - protein, 7 aa, synthetic construct
  REGION 1..7: note = Synthetic: AZD 7789 / PD-1 / VL CDR2
LTSNRAT

SEQ ID NO: 12 - protein, 7 aa, synthetic construct
  REGION 1..7: note = Synthetic: AZD 7789 / PD-1 / VL CDR3
QQWSSNP

SEQ ID NO: 13 - protein, 11 aa, synthetic construct
  REGION 1..11: note = Synthetic: TIM-3 (#62) / VL CDR3
QVLDRRSDHWL

SEQ ID NO: 14 - protein, 122 aa, synthetic construct
  REGION 1..122: note = Synthetic: AZD 7789 / TIM-3 / VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGSYGTYYGNYFEYWGQGTLVTVSS

SEQ ID NO: 15 - protein, 452 aa, synthetic construct
  REGION 1..452: note = Synthetic: AZD 7789 / TIM-3 / HC
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGSYGTYYGNYFEYWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 108 aa, synthetic construct
  REGION 1..108: note = Synthetic: TIM3 (#62) Variable Light VL
QTVLTQPPSVSVAPGKTASISCGGDNIGGKSVHWYQQKPGQAPVLVIYYDSDRPSGIPQRFSGSNSGNTATLTIHRVEAGDEADYYCQVLDRRSDHWLFGGGTKLTVL

SEQ ID NO: 17 - protein, 108 aa, synthetic construct
  REGION 1..108: note = Synthetic: AZD 7789 / TIM-3 / VL
SYVLTQPPSVSVAPGKTARITCGGDNIGGKSVHWYQQKPGQAPVLVIYYDSDRPSGIPERFSGSNSGNTATLTISRVEAGDEADYYCQVLDRRSDHFLFGGGTKLTVL

SEQ ID NO: 18 - protein, 214 aa, synthetic construct
  REGION 1..214: note = Synthetic: AZD 7789 / TIM-3 / LC
SYVLTQPPSVSVAPGKTARITCGGDNIGGKSVHWYQQKPGQAPVLVIYYDSDRPSGIPERFSGSNSGNTATLTISRVEAGDEADYYCQVLDRRSDHFLFGGGTKLTVLGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 19 - protein, 122 aa, synthetic construct
  REGION 1..122: note = Synthetic: AZD / 7789 / PD-1 / VH
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSS

SEQ ID NO: 20 - protein, 452 aa, synthetic construct
  REGION 1..452: note = Synthetic: AZD 7789 / PD-1 / HC
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 112 aa, synthetic construct
  REGION 1..112: note = Synthetic: AZD 7789 / PD-1 / VL
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIK

SEQ ID NO: 22 - protein, 219 aa, synthetic construct
  REGION 1..219: note = Synthetic: AZD 7789 / PD-1 / LC
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 710 aa, synthetic construct
  REGION 1..710: note = Synthetic: TIM-3 Heavy Chain
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVWDVSHEDPEVKFNWYVDGVEVHNAKTKPREEQYNSTYRWSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLLESGGGLVQPGGSLRLSCAASGFTFSSYAMSWVRQAPGKCLEWVSAISGSGGSTYYADSVKGRFTISRDNSKNTLYLQMNSLRAEDTAVYYCARGSYGTYYGNYFEYWGQGTLVTVSSGGGGSGGGGSGGGGSGGGGSSYVLTQPPSVSVAPGKTARITCGGDNIGGKSVHWYQQKPGQAPVLVIYYDSDRPSGIPERFSGSNSGNTATLTISRVEAGDEADYYCQVLDRRSDHFLFGCGTKLTVL

SEQ ID NO: 24 - protein, 218 aa, synthetic construct
  REGION 1..218: note = Synthetic: TIM-3 Light Chain / Variable Region
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 720 aa, synthetic construct
  REGION 1..720: note = Synthetic: TIM-3 Heavy Chain
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVWDVSHEDPEVKFNWYVDGVEVHNAKTKPREEQYNSTYRWSVLTVLHQDWLNGKEYKCKVSNKALPASIEKTISKAKGQPREPQVYTLPPSREEMTKNQVSLTCLVKGFYPSDIAVEWESNGGGGSGGGGSEVQLLESGGGLVQPGGSLRLSCAASGFTFSSYAMSWVRQAPGKCLEWVSAISGSGGSTYYADSVKGRFTISRDNSKNTLYLQMNSLRAEDTAVYYCARGSYGTYYGNYFEYWGQGTLVTVSSGGGGSGGGGSGGGGSGGGGSSYVLTQPPSVSVAPGKTARITCGGDNIGGKSVHWYQQKPGQAPVLVIYYDSDRPSGIPERFSGSNSGNTATLTISRVEAGDEADYYCQVLDRRSDHFLFGCGTKLTVLGGGGSGGGGSGQPENNYKTTPPVLDSDGSFFLYSKLTVDKSRWQQGNVFSCSVMHEALHNHYTQKSLSLSPGK

SEQ ID NO: 26 - protein, 218 aa, synthetic construct; identical to SEQ ID NO: 24
  REGION 1..218: note = Synthetic: TIM-3 Light Chain

SEQ ID NO: 27 - protein, 122 aa, synthetic construct
  REGION 1..122: note = Synthetic: TIM3 (#62) Variable Heavy VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGSYGTYYGNYFEYWGRGTLVTVSS

SEQ ID NO: 28 - protein, 288 aa, Homo sapiens
  REGION 1..288: note = Amino acid sequence of human PD-1 protein
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 29 - protein, 109 aa, Homo sapiens
  REGION 1..109: note = Human TIM-3 IgV domain
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIK

SEQ ID NO: 30 - protein, 301 aa, Homo sapiens
  REGION 1..301: note = Human TIM-3 protein
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP